(19) United States
(12) Patent Application Publication
(10) Pub. No.: US 2025/0009767 A1
(43) Pub. Date: Jan. 9, 2025

(54) Adhesive Oral Disc For Sustained Release Of Cannabinoids

(71) Applicant: Fertin Pharma A/S, Vejle (DK)

(72) Inventors: Helle Wittorff, Vejle Ost (DK); Sanne Skov Jensen, Fredericia (DK); Gitte Nykjær Nikolajsen, Viby J (DK); Gordon Findlay Dawson, Genolier (CH)

(73) Assignee: Fertin Pharma A/S, Vejle (DK)

(21) Appl. No.: 18/765,566

(22) Filed: Jul. 8, 2024

(30) Foreign Application Priority Data
Jul. 7, 2023 (DK) PA202370372
Jul. 7, 2023 (DK) PA202370373
Nov. 7, 2023 (DK) PA202330319

(51) Int. Cl. A61K 31/00; A61K 9/00; A61K 9/20; A61K 45/06

(52) U.S. Cl. CPC A61K 31/658; A61K 9/0056; A61K 9/006; A61K 9/2013; A61K 9/2018; A61K 9/205; A61K 9/2054; A61K 9/2086; A61K 45/06

(57) ABSTRACT

An adhesive oral disc for sustained release of cannabinoids is presented. The disc includes one or more cannabinoids and is composed of at least a mucosal contacting layer having a mucosal contacting surface operable to be fixed to the gingiva of a person in need of alleviation or treatment of a medical condition, where the mucosal contacting layer includes one or more mucoadhesive polymers allowing the adhesive oral disc to adhere to the gingiva for an extended period of time. Further, the disc includes a non-mucoadhesive module fused to the mucosal contacting layer, where the non-mucoadhesive module includes a solid tablet composition allowing the non-mucoadhesive module to dissolve within an extended period of time upon adhesion of the adhesive oral disc to the gingiva. The invention is particularly useful for alleviation or treatment of ulcers.

### FIELD OF INVENTION

The present invention relates to an adhesive oral disc suitable for oral administration of cannabinoids. Particularly, the invention is useful for sustained release of cannabinoids and for alleviation or treatment of medical indications, such as oral medical indications.

### BACKGROUND

Cannabinoids are a group of chemicals found in Cannabis sativa, Cannabis indica, Cannabis ruderalis, Marijuana plant and related plant species. They are known to activate cannabinoid receptors (CB1 and CB2). These chemicals are also produced endogenously in humans and other animals. Cannabinoids are cyclic molecules exhibiting particular properties such as being lipophilic, have the ability to easily cross the blood-brain barrier, and having low toxicity.

Cannabis sativa contains more than 400 chemicals and approximately 120 cannabinoids, the active constituents of cannabis, including tetrahydrocannabinol (THC), cannabidiol (CBD), cannabinol (CBN), tetrahydrocannabivarin (THCV) and cannabigerol (CBG). Pharmacologically, the principal psychoactive constituent of cannabis is tetrahydrocannabinol (THC), which is used for treating a wide range of medical conditions, including glaucoma, AIDS wasting, neuropathic pain, treatment of spasticity associated with multiple sclerosis, fibromyalgia, and chemotherapy-induced nausea. THC is also effective in the treatment of allergies, inflammation, infection, depression, migraine, bipolar disorders, anxiety disorder, drug dependency and drug withdrawal syndromes.

Various attempts have been made to formulate tablets for oral delivery of cannabinoids, such as cannabidiol (CBD). However, various challenges are associated with providing prolonged delivery of the active and at the same time keep a safe and convenient delivery. This is particularly important when the medical indication is present as a local medical indication in the oral cavity, such as ulcers, gingivitis, and the like. In these cases, it may be advantageous to provide a local delivery of cannabinoids, such as cannabidiol, with a sustained release of the active that is targeted to the medical indication. Various conventional tablets have been provided in the art that serve to release cannabinoids to the oral cavity in a broad sense. However, these tablets generally fail to deliver cannabinoids to local targets in the oral cavity with an improved release time, such as a sustained release time, to obtain optimal conditions for alleviation or treatment of medical indications, such as ulcers.

In some cases, various oral disorders may be found as local disorders in the oral cavity, such as ulcers. By targeting alleviation or treatment to these places in the oral cavity, the active may be applied in an increased concentration and/or applied with a higher precision in terms of release time suitable for alleviation or treatment of the condition.

Furthermore, it is important that a formulation is provided that may also help in obtaining increased convenience and effectiveness. In general, less attention is given in the prior art on the impact of the formulation of the oral delivery platform for the sensorics properties of oral cannabinoid delivery. These properties are both relevant from a convenience perspective for oral administration, but certainly also in order to support an appropriate delivery of cannabinoids and avoid adverse side effects of cannabinoids.

Hence, there is a need in the prior art for improved delivery vehicles that solve the above-referenced challenges and problems of the prior art. In particular, there is a need in the prior art for new delivery vehicles that support appropriate sustained delivery of cannabinoids combined with beneficial sensorics properties. Also, there is a need in the prior art for new delivery vehicles that support local administration of cannabinoids combined with optimized release time of the actives.

### SUMMARY

Accordingly, in a first aspect of the invention, there is provided an adhesive oral disc for sustained release of cannabinoids, the disc comprising one or more cannabinoids and being composed of at least: a mucosal contacting layer having a mucosal contacting surface operable to be fixed to the gingiva of a person in need of alleviation or treatment of a medical condition, the mucosal contacting layer comprising one or more mucoadhesive polymers allowing the adhesive oral disc to adhere to the gingiva for an extended period of time; and a non-mucoadhesive module fused to the mucosal contacting layer, the non-mucoadhesive module comprising a solid tablet composition allowing the non-mucoadhesive module to dissolve within an extended period of time upon adhesion of the adhesive oral disc to the gingiva.

In a second aspect, there is provided an adhesive oral disc for sustained release of cannabinoids, the disc comprising one or more cannabinoids and being composed of at least: a mucosal contacting layer having a mucosal contacting surface operable to be fixed to the gingiva of a person in need of alleviation or treatment of a medical condition, the mucosal contacting layer comprising one or more mucoadhesive polymers allowing the adhesive oral disc to adhere to the gingiva for an extended period of time; and a non-mucoadhesive module fused to the mucosal contacting layer, the non-mucoadhesive module being free of mucoadhesive polymers and comprising a solid tablet composition allowing the non-mucoadhesive module to dissolve within an extended period of time upon adhesion of the adhesive oral disc to the gingiva.

In a third aspect, there is provided an adhesive oral disc for sustained release of cannabinoids, the disc comprising: one or more cannabinoids; one or more sugar alcohol particles in an amount of at least 40% by weight of the adhesive oral disc: one or more mucoadhesive polymers in an amount of at least 5% by weight of the adhesive oral disc allowing the adhesive oral disc to adhere to the gingiva of a person in need of alleviation or treatment of a medical condition for an extended period of time.

Importantly, the present invention may not necessarily be provided in a so-called “multi-layered” configuration, i.e., comprising one mucosal contacting layer and one non-mucoadhesive module. The invention may also be provided in a configuration without these layers and/or modules, e.g., a configuration with only one layer or module. In some embodiments according to the invention, this configurations provided certain advantages that was not foreseen by the inventors to be possible.

In a fourth aspect, there is provided an adhesive oral disc for sustained release of cannabinoids, the disc comprising one or more cannabinoids and being composed of at least: a mucosal contacting layer having a mucosal contacting surface operable to be fixed to the gingiva of a person in need of alleviation or treatment of a medical condition, the mucosal contacting layer comprising one or more mucoadhesive polymers allowing the adhesive oral disc to adhere to the gingiva for an extended period of time; and a non-mucoadhesive module fused to the mucosal contacting layer, the non-mucoadhesive module comprising a solid tablet composition, including one or more binders, allowing the non-mucoadhesive module to dissolve within an extended period of time upon adhesion of the adhesive oral disc to the gingiva.

As used herein, the term “dissolve” is the process where a solid substance enters a solvent (oral saliva) to yield a solution. Unless otherwise stated, dissolving implies a full dissolving of the compound in question.

It is noted within the present context that the term “sustained release” refers to release of the cannabinoids over a prolonged period of time, in particular over period of at least 30 minutes, such as at least 45 minutes, such as at least 1 hour.

One of the advantages of the present invention is that sustained release and/or delivery of cannabinoids, such as cannabidiol, may be obtained. Another advantage of the present invention is that a local delivery of cannabinoids may be obtained. Due to the mucosal contacting layer, the oral disc may be targeted to the intended position between the gum and the lip, such as adherence to the gingiva.

In certain embodiments, the non-mucoadhesive module does not comprise mucoadhesive polymers. This is particularly useful to avoid adherence of the disc to the lips, which may provide discomfort and challenges for the disc to work properly. Without being bound by theory, it is contemplated that if the non-mucoadhesive module comprises mucoadhesive polymers, particularly in a relative high concentration, it may seriously affect release of cannabinoids from the disc and/or affect properly positioning of the disc to the target area. If the non-mucoadhesive module adheres to the lip apart from the oral disc adhering to the gingiva, it may seriously affect the usability of the oral disc. However, non-substantial amounts of mucoadhesive polymers may not result in these disadvantages but may in some embodiments be an advantage.

In certain embodiments, the non-mucoadhesive module comprises binders, such as cellulose derivative binders. This is particularly advantageous in order to minimize the use of mucoadhesives that my otherwise be applied in the non-mucoadhesive module. To the surprise of the inventors, it was seen that the use of binders, such as cellulose derivative binders, were possible to apply instead of mucoadhesives. These binders may greatly enhance the texture of the non-mucoadhesive module, including proper dissolution of the non-mucoadhesive module.

Furthermore, due to the effective adherence facilitated by the mucosal contacting layer, a convenient user experience may be provided. Since the disc is attached to the gingiva, the concentration of cannabinoids, such as cannabidiol may be higher locally compared to tablets, where a cannabinoids are released to the whole oral cavity. This both gives room for an increased efficiency and in turn may give room for providing a smaller disc compared to cannabinoid tablets with a non-focused delivery of cannabinoids to the oral mucosa.

Typically, smaller tablets positioned between the gum and the lip, such as on the gingiva, may easily detach from their intended position and may thus be unintentionally chewed or swallowed or even fall out of the mouth. Such unintended detachment may also result in excessive cannabinoids in the mouth and throat due to faster than intended release of cannabinoids followed by swallowing. In turn, this also leads to inefficient utilization of cannabinoids intended for local, oral action.

When the oral delivery vehicle is provided in a relatively smaller size compared to conventional cannabinoid tablets and adhered to the gingiva, it may be possible for a user to drink or even eat in the meantime without affecting the positioning of the oral disc and without affecting release of cannabinoids from the oral disc to a substantial degree. These advantages may be obtained without requiring any subsequent user action, such as special use, removal of residual product etc.

In the present context, when the non-mucoadhesive module is mentioned to be “fused to the mucosal contacting layer”, the intended meaning is that these two parts are fixed to each other during storage and during use. This may be done by compressing or pressing the two parts together in a tableting machine, or this may be done by first compressing one or both parts of the oral disc and thereafter gluing the two parts together with appropriate means. According to an advantageous embodiment of the invention, the oral disc is a compressed oral disc, where both parts are compressed.

In the present context, the term “compressed” refers to formed by compression from a plurality of particles and/or granules, such as by means of a tableting machine, such as e.g. a rotary press. The oral disc of the above embodiment is a compressed oral disc formed by compression of at least a first powdered composition and a second powdered composition to give the mucosal contacting layer as a “first layer” and the non-mucoadhesive module as a “second layer”, respectively. Accordingly, in the present context, the wording “first layer” generally refers to the mucosal contacting layer, whereas the wording “second layer” generally refers to the non-mucoadhesive module of the oral disc.

Typically, the oral disc may be made in a multi-step process, by compressing the “first layer” or “second module” first, then sequentially compressing the other layer(s) or module(s) to obtain a multilayer oral disc. When the oral disc is a two-layered tablet, it may be made in a two-step process, by compressing the “first layer” or “second module”, then compressing the other layer or module to obtain the two-layer oral disc.

In the present context, the term “layer” is generally applied for the mucosal contacting layer due to the relatively thinner thickness of this “layer” compared to the “module” of the non-mucoadhesive module, which is generally thicker. Generally, however, the two parts of the oral disc may be considered “layers” but may also be considered “modules” dependent on the context. Typically, the two parts of the oral disc has an appearance of a layered tablet. When applying the term “oral disc”, the intention is to direct the attention to the thickness of the oral vehicle, i.e., that an “oral disc” generally is considered to be thinner compared to an “oral tablet”.

For the avoidance of doubt, in the present context a “layer” or “module” is not to be understood as an outer “coating”. In some embodiments, a “coating” may be applied on top of the adhesive oral disc. In some embodiments, the adhesive oral disc does not comprise an outer coating.

In an embodiment of the invention, the mucosal contacting layer is compressed. In an embodiment of the invention, the non-mucoadhesive module is compressed. According to an advantageous embodiment of the invention, the oral disc is composed of a plurality of compressed particles. In an embodiment of the invention, the mucosal contacting layer is composed of a plurality of compressed particles. In an embodiment of the invention, the non-mucoadhesive module is composed of a plurality of compressed particles. According to an advantageous embodiment of the invention, the mucosal contacting layer and the non-mucoadhesive module are fused by compression.

In some embodiments of the invention, the solid tablet composition comprises one or more separate binders, such as one or more separate cellulose derivative binders. This particular embodiments excludes for instance binders such as lactose binders or other binders that do not originate from cellulose. These other binders may be less beneficial due to the nature of these binders.

It was a surprise to the inventors that the addition of one or more binders contributed to the advantageous improvements of the oral disc as the case were, particularly in the non-mucoadhesive module. The addition of one or more separate binders was seen to make it possible to compensate for the addition of mucoadhesive polymers, particularly in the non-mucoadhesive module. Hence, by adding one or more separate binders, the presence of mucoadhesive polymers could be lowered, particularly in the non-mucoadhesive module. One of the advantages was seen to be an improved texture and dissolution profile of the non-mucoadhesive module. In terms of the mucoadhesive contacting layer, the same adherence could be maintained even by lowering the amount of mucoadhesive, when one or more separate binders were added.

In the present context, when the one or more binders are mentioned to be “separate”, the intention is to exclude binders that are otherwise comprised in particles with other ingredients, such as sugar alcohols.

For instance, binders may be applied in granulated sugar alcohol particles together with sugar alcohol. These are not included in within the meaning of “separate binders” of the present invention. The term “separate binders” also excludes binders otherwise applied in wet or dry granulation. As used herein, the term “binder” refers to an ingredient promoting cohesiveness to the powder composition during the manufacturing process and thereby facilitating production of layers/modules and thereby discs with a desirable mechanical strength. Binder may advantageously be included in the non-mucoadhesive module, whereby a desirable cohesiveness during tableting is achieved.

The term “solid tablet composition” is intended to mean that the components of the non-mucoadhesive module is composed of particulate material. In some embodiments, the non-mucoadhesive module comprises more than 80% by weight of a solid tablet composition in the module, such as more than 90%, such as more than 95%, such as more than 98%, such as 100%

In some embodiments of the invention, the solid tablet composition comprises one or more separate binders in an amount of 1 to 15% by weight of the non-mucoadhesive module. In some embodiments of the invention, the solid tablet composition comprises one or more separate binders in an amount of 2 to 10% by weight of the non-mucoadhesive module. In some embodiments of the invention, the solid tablet composition comprises one or more separate binders in an amount of 2 to 10% by weight of the non-mucoadhesive module. In some embodiments of the invention, the solid tablet composition comprises one or more separate binders in an amount of 2 to 8% by weight of the non-mucoadhesive module. In some embodiments of the invention, the solid tablet composition comprises one or more separate binders in an amount of 4 to 8% by weight of the non-mucoadhesive module.

In some embodiments of the invention, the solid tablet composition comprises one or more separate binders in an amount of more than 1% by weight of the non-mucoadhesive module. In some embodiments of the invention, the solid tablet composition comprises one or more separate binders in an amount of more than 2 10% by weight of the non-mucoadhesive module. In some embodiments of the invention, the solid tablet composition comprises one or more separate binders in an amount of more than 4% by weight of the non-mucoadhesive module.

In some embodiments of the invention, the solid tablet composition comprises one or more separate binders in an amount of less than 15% by weight of the non-mucoadhesive module. In some embodiments of the invention, the solid tablet composition comprises one or more separate binders in an amount of less than 10% by weight of the non-mucoadhesive module. In some embodiments of the invention, the solid tablet composition comprises one or more separate binders in an amount of less than 8% by weight of the non-mucoadhesive module. In some embodiments of the invention, the solid tablet composition comprises one or more separate binders in an amount of less than 6% by weight of the non-mucoadhesive module. In some embodiments of the invention, the solid tablet composition comprises one or more separate binders in an amount of less than 4% by weight of the non-mucoadhesive module. In some embodiments of the invention, the solid tablet composition comprises one or more separate binders in an amount of less than 2% by weight of the non-mucoadhesive module. In some embodiments of the invention, the solid tablet composition comprises one or more separate binders in an amount of less than 1% by weight of the non-mucoadhesive module. In some embodiments of the invention, the solid tablet composition comprises does not comprise one or more separate binders in the non-mucoadhesive module.

In some embodiments of the invention, the solid tablet composition comprises one or more separate binders selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

In some embodiments of the invention, the solid tablet composition comprises one or more separate cellulose derivative binders selected from the group consisting of carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

In some embodiments of the invention, the solid tablet composition comprises one or more separate binders selected from the group consisting of hydroxypropyl cellulose, hydroxypropyl methylcellulose, and any combination thereof.

In some embodiments of the invention, the solid tablet composition comprises hydroxypropyl cellulose (HPC).

In some embodiments of the invention, the solid tablet composition comprises hydroxypropyl methylcellulose (HPMC).

In some embodiments of the invention, the solid tablet composition comprises one or more sugar alcohol particles.

In some embodiments of the invention, the solid tablet composition comprises one or more sugar alcohol particles in an amount of at least 40% by weight of the non-mucoadhesive module. In some embodiments of the invention, the solid tablet composition comprises one or more sugar alcohol particles in an amount of at least 50% by weight of the non-mucoadhesive module. In some embodiments of the invention, the solid tablet composition comprises one or more sugar alcohol particles in an amount of at least 60% by weight of the non-mucoadhesive module. In some embodiments of the invention, the solid tablet composition comprises one or more sugar alcohol particles in an amount of at least 70% by weight of the non-mucoadhesive module. In some embodiments of the invention, the solid tablet composition comprises one or more sugar alcohol particles in an amount of at least 80% by weight of the non-mucoadhesive module.

In some embodiments of the invention, the solid tablet composition comprises one or more sugar alcohol particles in an amount of 40 to 98% by weight of the non-mucoadhesive module. In some embodiments of the invention, the solid tablet composition comprises one or more sugar alcohol particles in an amount of 45 to 95% by weight of the non-mucoadhesive module. In some embodiments of the invention, the solid tablet composition comprises one or more sugar alcohol particles in an amount of 50 to 90% by weight of the non-mucoadhesive module.

In some embodiments of the invention, the solid tablet composition comprises one or more sugar alcohol particles selected from the group consisting of xylitol, maltitol, mannitol, erythritol, isomalt, sorbitol, lactitol, and any combination thereof.

The presence of one or more sugar alcohols is particularly advantageous according to the invention. Especially, the presence of one or more sugar alcohols may accommodate the dissolution time of the module.

In some embodiments of the invention, the solid tablet composition does not comprise a flavoring agent.

In some embodiments of the invention, the oral disc does not comprise an edible alcohol, such as not comprising an edible alcohol in the non-mucoadhesive module. An edible alcohol, if present, may detriment the overall composition.

In the present context, the term “edible alcohol” is intended to mean alcohols liquid at 25 Degree Celsius, such as ethanol, methanol, propylene glycol, glycerol, or the like.

In some embodiments of the invention, the solid tablet composition comprises one or more cannabinoids. In some embodiments of the invention, the solid tablet composition comprises one or more cannabinoids in an amount of 1 to 100 mg. In some embodiments of the invention, the solid tablet composition comprises one or more cannabinoids in an amount of 2 to 50 mg. In some embodiments of the invention, the solid tablet composition comprises one or more cannabinoids in an amount of 5 to 30 mg. In some embodiments of the invention, the solid tablet composition comprises one or more cannabinoids in an amount of 10 to 20 mg, which is particularly advantageous for alleviation or treatment of ulcers.

In some embodiments of the invention, the one or more mucoadhesive polymers is present in an amount of at least 20% by weight of the mucosal contacting layer. In some embodiments of the invention, the one or more mucoadhesive polymers is present in an amount of at least 40% by weight of the mucosal contacting layer. In some embodiments of the invention, the one or more mucoadhesive polymers is present in an amount of at least 60% by weight of the mucosal contacting layer. These amounts are intended to refer to the amount of mucoadhesive polymers in the mucosal contacting layer. In the present context in order for the “mucosal contacting layer” to be adhesive, a substantial amount of mucoadhesive polymers are to be present in the layer.

In some embodiments of the invention, the one or more mucoadhesive polymers is present in an amount of 20 to 95% by weight of the mucosal contacting layer. In some embodiments of the invention, the one or more mucoadhesive polymers is present in an amount of 25 to 90% by weight of the mucosal contacting layer. In some embodiments of the invention, the one or more mucoadhesive polymers is present in an amount of 30 to 85% by weight of the mucosal contacting layer. In some embodiments of the invention, the one or more mucoadhesive polymers is present in an amount of 35 to 80% by weight of the mucosal contacting layer. In some embodiments of the invention, the one or more mucoadhesive polymers is present in an amount of 40 to 80% by weight of the mucosal contacting layer. In some embodiments of the invention, the one or more mucoadhesive polymers is present in an amount of 45 to 80% by weight of the mucosal contacting layer. In some embodiments of the invention, the one or more mucoadhesive polymers is present in an amount of 50 to 80% by weight of the mucosal contacting layer. In some embodiments of the invention, the one or more mucoadhesive polymers is present in an amount of 60 to 80% by weight of the mucosal contacting layer.

An advantage of the above embodiments may be that the mucoadhesive provides a gel like structure at the end of dissolution. To some users the gel like structure may be experienced as preferable compared to mucoadhesives not comprising cellulose derivatives, as the latter may provide a more rigid structure during dissolution. Without being bound by theory it is contemplated that the gel like structure occurs as a result of excessive swelling of the cellulose derivative upon prolonged exposure to saliva.

In some embodiments of the invention, the one or more mucoadhesive polymers is further present in the non-mucoadhesive module in an amount of less than 20% by weight of the non-mucoadhesive module.

In some embodiments of the invention, the one or more mucoadhesive polymers is further present in the non-mucoadhesive module in an amount of less than 15% by weight of the non-mucoadhesive module.

In some embodiments of the invention, the one or more mucoadhesive polymers is further present in the non-mucoadhesive module in an amount of less than 10% by weight of the non-mucoadhesive module.

In some embodiments of the invention, the one or more mucoadhesive polymers is further present in the non-mucoadhesive module in an amount of less than 5% by weight of the non-mucoadhesive module.

In some embodiments of the invention, the one or more mucoadhesive polymers is further present in the non-mucoadhesive module in an amount of 1 to 20% by weight of the non-mucoadhesive module. In some embodiments of the invention, the one or more mucoadhesive polymers is further present in the non-mucoadhesive module in an amount of 1 to 10% by weight of the non-mucoadhesive module. In some embodiments of the invention, the one or more mucoadhesive polymers is further present in the non-mucoadhesive module in an amount of 1 to 5% by weight of the non-mucoadhesive module.

In some embodiments of the invention, the one or more mucoadhesive polymers is not present in the non-mucoadhesive module. In some embodiments of the invention, the one or more mucoadhesive polymers is substantially not present in the non-mucoadhesive module.

In some embodiments of the invention, the one or more mucoadhesive polymers is selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, dextran, guar gum, polyvinyl pyrrolidone (PVP), gelatin, casein, acrylic acid polymers (carbomers), acrylic acid esters, acrylic acid copolymers, polyethylene oxide, and any combination thereof.

In some embodiments of the invention, the one or more mucoadhesive polymers is selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, dextran, guar gum, polyvinyl pyrrolidone (PVP), gelatin, casein, acrylic acid polymers (carbomers), acrylic acid esters, acrylic acid copolymers, and any combination thereof.

In an embodiment of the invention, the mucoadhesive comprises a natural gum.

In an embodiment of the invention, the mucoadhesive consists of a natural gum.

In some embodiments of the invention, the one or more mucoadhesive polymers comprises natural gums selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, and combinations thereof. In some embodiments of the invention, the one or more mucoadhesive polymers comprises gum Arabic.

In some embodiments of the invention, the one or more mucoadhesive polymers comprises synthetic mucoadhesive (hydrophilic adhesives) selected from the group consisting of polyvinyl pyrrolidone (PVP), acrylic acid polymers (carbomers), acrylic acid esters, acrylic acid copolymers, and any combination thereof.

In some embodiments of the invention, the one or more mucoadhesive polymers comprises a hydrophilic polymer selected from the group consisting of polyvinyl pyrrolidone (PVP), acrylic acid polymers (carbomers), co-polymers of polyacrylic acid cross-linked with divinyl glycol and salts thereof (polycarbophil), copolymers of monoalkyl esters of poly(methyl vinyl ether/maleic acid) (Gantrez polymers), acrylic acid esters, acrylic acid copolymers, and any combination thereof.

In some embodiments of the invention, the one or more mucoadhesive polymers comprises polyvinyl pyrrolidone (PVP). In some embodiments of the invention, the one or more mucoadhesive polymers comprises acrylic acid polymers (carbomers). In some embodiments of the invention, the one or more mucoadhesive polymers comprises co-polymers of polyacrylic acid cross-linked with divinyl glycol and salts thereof (polycarbophil). In some embodiments of the invention, the one or more mucoadhesive polymers comprises copolymers of monoalkyl esters of poly(methyl vinyl ether/maleic acid) (Gantrez polymers). In some embodiments of the invention, the one or more mucoadhesive polymers comprises acrylic acid esters. In some embodiments of the invention, the one or more mucoadhesive polymers comprises acid copolymers.

In some embodiments of the invention, the one or more mucoadhesive polymers comprises an ionic mucoadhesive. As used herein, the term “ionic mucoadhesive” refers to ionizable polysaccharide mucoadhesives, sometimes also referred to as polyelectrolytes. Thus, ionic mucoadhesive does not include e.g. polyvinyl pyrrolidone. An advantage of the above embodiment may be that the release profile of the active ingredient may be altered/modified.

In some embodiments of the invention, the one or more mucoadhesive polymers comprises a mucoadhesive selected from the group consisting of xanthan gum, gellan gum, gum arabic, alginic acid, alginate, carrageenan, agar, and any combination thereof. In some embodiments of the invention, the one or more mucoadhesive polymers comprises xanthan gum and/or gum arabic. In some embodiments of the invention, the one or more mucoadhesive polymers comprises gum arabic. In some embodiments of the invention, the one or more mucoadhesive polymers comprises xanthan gum.

In some embodiments of the invention, the one or more mucoadhesive polymers comprises sodium alginate.

In some embodiments of the invention, the one or more mucoadhesive polymers is added as separate components in the adhesive oral disc.

In some embodiments of the invention, the one or more mucoadhesive polymers is added as separate components from the one or more cannabinoids in the adhesive oral disc.

In some embodiments of the invention, the one or more cannabinoids is not bound to the one or more mucoadhesive polymers in the adhesive oral disc.

In some embodiments of the invention, the mucosal contacting layer comprises one or more separate binders. In some embodiments of the invention, the mucosal contacting layer comprises one or more separate cellulose derivative binders.

An advantage of the above embodiment may be that the mucoadhesive provides a gel like structure at the end of dissolution. To some users the gel like structure may be experienced as preferable compared to mucoadhesives not comprising cellulose derivatives, as the latter may provide a more rigid structure during dissolution. Without being bound by theory it is contemplated that the gel like structure occurs as a result of excessive swelling of the cellulose derivative upon prolonged exposure to saliva.

In some embodiments of the invention, the mucosal contacting layer comprises one or more separate binders comprising one or more separate cellulose derivative binders selected from the group consisting of hydroxypropyl cellulose (HPC), hydroxypropyl methylcellulose (HPMC), hydroxy ethylcellulose, ethylcellulose (EC), carboxymethyl cellulose (CMC), and salts thereof, and any combination thereof.

In some embodiments of the invention, the mucosal contacting layer comprises one or more separate binders selected from the group consisting of hydroxypropyl cellulose, hydroxypropyl methylcellulose, and any combination thereof.

In some embodiments of the invention, the mucosal contacting layer comprises one or more separate binders comprising hydroxypropyl cellulose (HPC). In some embodiments of the invention, the mucosal contacting layer comprises one or more separate binders comprising hydroxypropyl methylcellulose (HPMC).

In some embodiments of the invention, the mucosal contacting layer comprises one or more separate binders in an amount of 1 to 15% by weight of the mucosal contacting layer. In some embodiments of the invention, the mucosal contacting layer comprises one or more separate binders in an amount of 2 to 10% by weight of the mucosal contacting layer. In some embodiments of the invention, the mucosal contacting layer comprises one or more separate binders in an amount of 4 to 8% by weight of the mucosal contacting layer.

In some embodiments of the invention, the mucosal contacting layer comprises one or more separate binders selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

In some embodiments of the invention, the mucosal contacting layer comprises one or more separate cellulose derivative binders selected from the group consisting of carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof. In some embodiments of the invention, the mucosal contacting layer comprises hydroxypropyl cellulose (HPC). In some embodiments of the invention, the mucosal contacting layer comprises hydroxypropyl methylcellulose (HPMC).

An advantage of the above embodiment may be that the mucoadhesive provides a gel like structure at the end of dissolution. To some users the gel like structure may be experienced as preferable compared to mucoadhesives not comprising cellulose derivatives, as the latter may provide a more rigid structure during dissolution. Without being bound by theory it is contemplated that the gel like structure occurs as a result of excessive swelling of the cellulose derivative upon prolonged exposure to saliva.

In an embodiment of the invention, the dissolution time of the above embodiments is in vivo dissolution time.

According to an embodiment of the invention, the in vivo dissolution time was measured by at least 6 trained assessors, the trained assessors abstaining from eating and drinking at least 30 minutes before initiation of any test, the disc was weighted and placed in the mouth, between the upper lip and the gum with the first facing the gum, where the in vivo dissolution time was registered as the time point where the oral disc dissolution was substantially complete.

In some embodiments of the invention, the mucosal contacting layer comprises one or more sugar alcohol particles. In some embodiments of the invention, the mucosal contacting layer comprises one or more sugar alcohol particles in an amount of 5 to 80% by weight of the mucosal contacting layer. In some embodiments of the invention, the mucosal contacting layer comprises one or more sugar alcohol particles in an amount of 10 to 80% by weight of the mucosal contacting layer. In some embodiments of the invention, the mucosal contacting layer comprises one or more sugar alcohol particles in an amount of 20 to 80% by weight of the mucosal contacting layer. In some embodiments of the invention, the mucosal contacting layer comprises one or more sugar alcohol particles in an amount of 30 to 80% by weight of the mucosal contacting layer. In some embodiments of the invention, the mucosal contacting layer comprises one or more sugar alcohol particles in an amount of 40 to 80% by weight of the mucosal contacting layer. In some embodiments of the invention, the mucosal contacting layer comprises one or more sugar alcohol particles in an amount of 50 to 80% by weight of the mucosal contacting layer.

In some embodiments of the invention, the mucosal contacting layer comprises one or more sugar alcohol particles in an amount of 10 to 60% by weight of the mucosal contacting layer. In some embodiments of the invention, the mucosal contacting layer comprises one or more sugar alcohol particles in an amount of 10 to 40% by weight of the mucosal contacting layer.

In some embodiments of the invention, the mucosal contacting layer comprises one or more sugar alcohol particles selected from the group consisting of xylitol, maltitol, mannitol, erythritol, isomalt, sorbitol, lactitol, and any combination thereof.

In some embodiments of the invention, the mucosal contacting layer comprises fillers.

In some embodiments of the invention, the mucosal contacting layer comprises fillers selected from the group consisting of calcium carbonate, magnesium carbonate, magnesium dihydroxide, and combinations thereof.

In some embodiments of the invention, the mucosal contacting layer does not comprise a flavoring agent.

In some embodiments of the invention, the mucosal contacting layer comprises one or more cannabinoids. In some embodiments of the invention, the mucosal contacting layer comprises one or more cannabinoids in an amount of 1 to 100 mg. In some embodiments of the invention, the mucosal contacting layer comprises one or more cannabinoids in an amount of 2 to 50 mg. In some embodiments of the invention, the mucosal contacting layer comprises one or more cannabinoids in an amount of 5 to 30 mg.

In some embodiments of the invention, the mucosal contacting layer does not comprise one or more cannabinoids.

In some embodiments of the invention, the mucosal contacting layer constitutes at least 10% by weight of the adhesive oral disc, such as at least 20% by weight of the adhesive oral disc, such as at least 30% by weight of the adhesive oral disc.

In some embodiments of the invention, the non-mucoadhesive module constitutes at least 40% by weight of the adhesive oral disc, such as at least 50 by weight of the adhesive oral disc, such as at least 60% by weight of the adhesive oral disc.

In some embodiments of the invention, the mucosal contacting layer constitutes at 10 to 50% by weight of the adhesive oral disc, such as 20 to 50% by weight of the adhesive oral disc, such as 20 to 40% by weight of the adhesive oral disc.

In some embodiments of the invention, the adhesive oral disc has a weight of at least 100 mg, such as at least 150 mg, such as at least 200 mg.

In some embodiments of the invention, the adhesive oral disc has a weight of no more than 600 mg, such as no more than 500 mg, such as no more than 400 mg.

In some embodiments of the invention, the mucosal contacting layer is substantially flat.

In some embodiments of the invention, the mucosal contacting layer is concave, dimple-shaped, donut-shaped, or the mucosal contacting surface of the mucosal contacting layer comprises one or more of these shapes.

In some embodiments of the invention, the mucosal contacting surface of the mucosal contacting layer comprises an inward depression that allows the layer to be fixed to the gingiva as a first mechanism by pressure difference.

In some embodiments of the invention, the mucosal contacting layer comprises an inward depression that allows the layer to be fixed to the gingiva as a first mechanism by pressure difference.

In some embodiments of the invention, the adhesive oral disc does not comprise a gel layer or cast film strip layer.

In some embodiments of the invention, the adhesive oral disc comprises less than 15% by weight of moisture. In some embodiments of the invention, the adhesive oral disc comprises less than 10% by weight of moisture. In some embodiments of the invention, the adhesive oral disc comprises less than 5% by weight of moisture.

In some embodiments of the invention, the mucosal contacting layer comprises less than 15% by weight of moisture. In some embodiments of the invention, the mucosal contacting layer comprises less than 10% by weight of moisture. In some embodiments of the invention, the mucosal contacting layer comprises less than 5% by weight of moisture.

In some embodiments of the invention, the mucosal contacting layer is a compressed layer.

In some embodiments of the invention, the non-mucoadhesive module is a compressed module.

In some embodiments of the invention, the mucosal contacting layer and the non-mucoadhesive module are fused by compression.

In some embodiments of the invention, the non-mucoadhesive module is not extending over any side surface of the mucosal contacting layer.

In some embodiments of the invention, the non-mucoadhesive module is not extending over side surfaces of the mucosal contacting layer, whereby a larger contact area is exposed to the oral mucosa compared to a configuration where the non-mucoadhesive module is extending over side surfaces of the mucosal contacting layer and only a bottom surface of the mucoadhesive module is exposed to the oral mucosa upon administration.

In some embodiments of the invention, the non-mucoadhesive module is positioned on top of the mucosal contacting layer.

In some embodiments of the invention, the adhesive oral disc has a dissolution time of at least 15 minutes, such as at least 20 minutes, such as at least 30 minutes, such as at least 45 minutes.

In some embodiments of the invention, the adhesive oral disc has a dissolution time of no more than 10 hours, such as 8 hours, such as 5 hours, such as 2.5 hours, such as no more than 2 hours, such as no more than 1.5 hours, such as no more than 1 hour.

In some embodiments of the invention, the adhesive oral disc has a dissolution time of between 15 minutes to 2.5 hours, such as 20 minutes to 1.5 hours, such as 30 minutes to 1 hour.

In some embodiments of the invention, the adhesive oral disc has a dissolution time of between 45 minutes to 2.5 hours. In some embodiments of the invention, the adhesive oral disc has a dissolution time of between 1 hour to 2 hours.

In some embodiments of the invention, the adhesive oral disc has a dissolution time of between 6 hours to 12 hours. In some embodiments of the invention, the adhesive oral disc has a dissolution time of between 7 hours to 11 hours. In some embodiments of the invention, the adhesive oral disc has a dissolution time of between 8 hours to 10 hours.

In some embodiments of the invention, the dissolution time of the mucosal contacting layer is substantially synchronized with the dissolution time of the non-mucoadhesive module.

In some embodiments of the invention, the extended period of time of adherence of the mucosal contacting layer of the adhesive oral disc to the gingiva corresponds to the extended period of time of dissolution of the non-mucoadhesive module of the adhesive oral disc.

In some embodiments of the invention, the one or more cannabinoids is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and combinations thereof.

In some embodiments of the invention, the one or more cannabinoids is selected from the group consisting of cannabidiol (CBD), cannabidiolic acid (CBDA), cannabidivarin (CBDV), and combinations thereof.

In some embodiments of the invention, the one or more cannabinoids is selected from the group consisting of tetrahydrocannabinol (THC), tetrahydrocannabinolic acid (THCA), tetrahydrocannabivarin (THCV), and combinations thereof.

In some embodiments of the invention, the one or more cannabinoids comprises cannabidiol (CBD).

In some embodiments of the invention, the one or more cannabinoids comprises an isolated cannabinoid.

In some embodiments of the invention, the one or more cannabinoids is an isolated cannabinoid.

In some embodiments of the invention, the one or more cannabinoids is a synthetic cannabinoid.

In some embodiments of the invention, the one or more cannabinoids is not a cannabinoid extract with a cannabinoid purity of less than 80%.

In some embodiments of the invention, the one or more cannabinoids is present as particles in a size of 400 to 1200 nanometers. In some embodiments of the invention, the one or more cannabinoids is present as particles in a size of 500 to 1000 nanometers.

In some embodiments of the invention, the one or more cannabinoids is present in the mucosal contacting layer.

In some embodiments of the invention, the one or more cannabinoids is present in the non-mucoadhesive module.

In some embodiments of the invention, the one or more cannabinoids is present in both the mucosal contacting layer and the non-mucoadhesive module.

In some embodiments of the invention, the one or more cannabinoids is not present in the mucosal contacting layer.

In some embodiments of the invention, the one or more cannabinoids is not present in the non-mucoadhesive module.

In some embodiments of the invention, the adhesive oral disc comprises one or more self-emulsifying systems.

In some embodiments of the invention, the adhesive oral disc comprises one or more liquid self-emulsifying systems.

In some embodiments of the invention, the adhesive oral disc comprises one or more liquid self-emulsifying systems comprising one or more surfactants having an HLB-value of more than 6.

In some embodiments of the invention, the adhesive oral disc comprises one or more liquid self-emulsifying systems comprising one or more surfactants selected from the group consisting of PEG-35 castor oil, PEG-6 oleoyl glycerides, PEG-6 linoleoyl glycerides, PEG-8 caprylic/capric glyceride, sorbitan monolaurate, sorbitan monooleate, polyoxyethylene (20) sorbitan monolaurate, polyoxyethylene (60) sorbitan monostearate, polyoxyethylene (80) sorbitan monooleate, lauroylpoloxyl-32 glycerides, stearoyl polyoxyl-32 glycerides, polyoxyl-32 stearate, propylene glycol mono laurate, propylene glycol di laurate, and mixtures and combinations thereof.

In some embodiments of the invention, the adhesive oral disc comprises one or more liquid self-emulsifying systems comprising one or more co-solvents.

In some embodiments of the invention, the adhesive oral disc comprises one or more liquid self-emulsifying systems comprising one or more co-solvents selected from the group consisting of polyglyceryl-3 dioleate, 1,2-propandiol, polyethylene glycol 300, polyethylene glycol 400, diethylene glycol monoethyl ether, and mixtures and combinations thereof.

In some embodiments of the invention, the adhesive oral disc comprises one or more liquid self-emulsifying systems comprising one or more solubilizers.

In some embodiments of the invention, the adhesive oral disc comprises one or more liquid self-emulsifying systems comprising one or more solubilizers selected from the group consisting of lauroylpoloxyl-32 glycerides; stearoyl polyoxyl-32 glycerides; Polyoxyl-32 stearate; synthetic copolymer of ethylene oxide (80) and propylene oxide (27); polyvinyl caprolactam-polyvinyl acetate-polyethylene glycol graft co-polymer; alpha-, beta- or gamma cyclodextrins and derivatives thereof; pea proteins (globulins, albumins, glutelins proteins); and mixtures and combinations thereof.

Due to the poor solubility of certain active ingredients in physiological fluids, it is an unmet need to solubilize cannabinoids upon mixture with the body physiological fluids to facilitate bio-absorption. To overcome low bioavailability, various lipid-based drug delivery systems and self-emulsifying systems have been developed. Lipid-based delivery systems and particularly self-emulsifying drug delivery systems (SEDDS) have been demonstrated to increase the solubility, dissolution and bioavailability of many insoluble active ingredients.

Particularly with respect to SEDDS, the formulation of the present invention may provide some clear benefits, both allowing a higher load of active ingredients and at the same time offer improved sensorics properties of the formulation during use. Other advantages are also present.

Importantly, the presence of SEDDS or at least a self-emulsifying agent was seen to act in synergy with the formulation of the present invention. The presence of SEDDS or at least a self-emulsifying agent was seen to further increase the uptake of the active ingredients through mucosal surfaces.

Accordingly, the synergy between the presence of SEDDS or at least a self-emulsifying agent and cannabinoids was a surprise to the inventors. Particularly, the potential of SEDDS to have a high load of active ingredients, further contributes to the synergy according to the invention.

In the present context, SEDDS is a solid or liquid dosage form comprising at least a surfactant and optionally a co-surfactant, characterized primarily in that said dosage form can form oil-in-water emulsion spontaneously in the oral cavity or at ambient temperature (referring generally to body temperature, namely 37° C.). When a SEDDS enters the mucosa, it is initially self-emulsified as emulsion droplets and rapidly dispersed. The resulting microparticulate of micrometer or nanometer level can penetrate into the mucous membrane, and the absorbed oil droplets enter the blood circulation, thereby significantly improving the bioavailability of the active ingredient.

In some embodiments of the invention, the one or more cannabinoids is bound to cyclodextrin, such as thiolated cyclodextrin.

In some embodiments of the invention, the one or more cannabinoids is comprised in a premix including one or more lipids.

In some embodiments of the invention, the one or more cannabinoids is comprised in a premix including one or more triglycerides.

In some embodiments of the invention, the one or more cannabinoids is comprised in a premix including one or more vegetable oils.

In some embodiments of the invention, the adhesive oral disc comprises one or more permeation enhancers.

According to an advantageous embodiment of the invention, the disc comprises a pH regulating agent, such as an alkaline pH regulating agent, such as an alkaline buffering agent. In an embodiment of the invention, the mucoadhesive contacting layer comprises a pH regulating agent. In an embodiment of the invention, the pH regulating agent of the mucoadhesive contacting layer is an alkaline pH regulating agent, such as an alkaline buffering agent. In an embodiment of the invention, the non-mucoadhesive module comprises a pH regulating agent. In an embodiment of the invention, the pH regulating agent of the non-mucoadhesive module is an alkaline pH regulating agent, such as an alkaline buffering agent.

In some embodiments of the invention, the oral disc further comprises one of more alkaline pH regulating agents, such as sodium carbonate or sodium hydrogen carbonate.

In some embodiments of the invention, the oral disc further comprises one of more acid neutralizing agents, such as calcium carbonate, sodium carbonate or sodium hydrogen carbonate.

In some embodiments of the invention, the oral disc further comprising one or more actives selected from the group consisting of chitosan, curcumin, propolis, hyaluronic acid, aloe vera, quercetin, and combinations thereof.

In some embodiments of the invention, the adhesive oral disc comprises a further module/layer.

In some embodiments of the invention, the adhesive oral disc comprises a second mucosal contacting layer.

In some embodiments of the invention, the adhesive oral disc further comprises an oral disintegrating tablet (ODT) module.

In some embodiments of the invention, the adhesive oral disc further comprises a fast disintegrating tablet (FDT) module.

In some embodiments of the invention, the adhesive oral disc comprises a further module/layer comprising a disintegrant.

In a further aspect of the invention, the oral disc according to the invention is for use in a method for the treatment or alleviation of a medical condition.

In a further aspect of the invention, the oral disc according to the invention is for use in a method for the treatment or alleviation of ulcers.

In a further aspect of the invention, the oral disc according to the invention is for use in a method for the treatment or alleviation of canker sores.

In a further aspect of the invention, the oral disc according to the invention is for use in a method for the treatment or alleviation of aphthous ulcers.

In a further aspect of the invention, the oral disc according to the invention is for use in a method for the treatment or alleviation of oral disorders.

### DETAILED DESCRIPTION

The invention will now be described in more details with respect to certain aspects and embodiments of the invention. These aspects and embodiments are intended to be understood in connection with the rest of the description, including the Summary of the Invention and the Examples of the invention.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

As used herein, the term “disintegrate” refers to a reduction of an object to components, fragments or particles. Disintegration time may be measured in vitro or in vivo. Unless otherwise stated, the in vitro measurements are carried out in accordance to European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

The term “tableted” or “compressed” is intended to mean that the oral disc composition is pressed in a tableting apparatus and mainly being composed of particulate matter. Although the terms imply a method step, in the present context, the terms are intended to mean the resulting tablet obtained in tableting a portion of particles. It is noted that an oral disc or tableted composition that is mentioned to comprise particles eventually is to be understood as particles that have been pressed together in a tableting step.

A “self-emulsifying agent” is an agent which will form an emulsion when presented with an alternate phase with a minimum energy requirement. In contrast, an emulsifying agent, as opposed to a self-emulsifying agent, is one requiring additional energy to form an emulsion.

As used herein, the term “release of cannabinoids” refers to the cannabinoids being made bioavailable, i.e. available for absorption over the mucous membrane in the oral cavity.

While some forms of cannabinoids require dissolution for being bioavailable, other forms may be readily absorbed into the body without dissolution.

As used herein, the term “pH regulating agent” refers to agents, which active adjust and regulates the pH value of the solution to which they have been added or are to be added. In the present context, pH regulating agents do not include active ingredients.

When referring to amounts of an ingredient by terms such as “less than”, “no more than”, this generally refers to the particular ingredient being absent or present in a range from trace amounts to the specified maximum amount.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. The flavors can be natural or synthetic flavors.

Typically, the oral disc may comprise ingredients selected from the group consisting of fillers, flavors, binders, disintegrants, hereunder super disintegrants, emulsifiers, antioxidants, pH regulating agents hereunder alkaline and acidic pH regulating agents, high intensity sweeteners, colors, glidants, lubricants, or any combination thereof.

In an advantageous embodiment of the invention, the oral disc comprises bulk sweetener as filler ingredient.

In an advantageous embodiment of the invention, the first layer comprises bulk sweetener as filler ingredient.

In an advantageous embodiment of the invention, the second layer comprises bulk sweetener as filler ingredient.

In an advantageous embodiment of the invention, the first and second layers comprise bulk sweetener as filler ingredient.

The oral disc may in addition to the at least one sugar alcohol of the first layer and the at least one sugar alcohol of the second layer, comprise different bulk sweeteners. Bulk sweeteners include sugar sweetener and/or sugarless sweetener.

Sugar sweeteners generally include, but are not limited to saccharide-containing components, such as sucrose, dextrose, maltose, saccharose, lactose, sorbose, dextrin, trehalose, D-tagatose, dried invert sugar, fructose, levulose, galactose, and the like, alone or in combination.

Sugarless sweeteners generally include but are not limited to sugar alcohols (also sometimes referred to as polyols) such as xylitol, maltitol, mannitol, erythritol, isomalt, sorbitol, lactitol, and combinations thereof.

Combinations of sugar and/or non-sugar sweeteners may be used in the oral disc.

The bulk sweeteners may often support the flavor profile of the oral disc.

In embodiment of the invention, bulk sweeteners may be supplemented with other usable fillers including as examples, magnesium and calcium carbonate, sodium sulphate, ground limestone, silicate compounds such as magnesium and aluminum silicate, kaolin and clay, aluminum oxide, silicon oxide, talc, titanium oxide, mono-, di- and tri-calcium phosphates, fibers, plant fibers, such as wheat fiber, oat fiber, pea fiber, and combinations thereof.

High intensity artificial sweetening agents can also be used in combination with the above bulk sweeteners. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (such as from about 0.02 to about 8% by weight).

In embodiments where the oral disc comprises flavor, different flavors may be used.

Usable flavors including as examples almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry, kiwi, tropical punch, tutti frutti, vanilla, or any combination thereof.

According to an embodiment of the invention, flavor may be used as taste masking for the cannabinoids and/or taste masking of the alkaline pH regulating agent. However, as outlined in other embodiments, flavors may preferably bee avoided in the non-mucoadhesive module. It may be a drawback to have flavors in the non-mucoadhesive module. Particularly flavors may interfere with an intended sustained release of cannabinoids. More particularly, flavors may in some cases promote saliva generation that may counteract sustained release of cannabinoids, such as in cases where accelerated dissolution of the non-mucoadhesive module takes place. Additionally, flavors may also increase the pain sensation experienced at the location of sensitive areas on the gingiva. This may be more pronounced for alleviation or treatment of medical indications, such as ulcers or gingivitis.

In an embodiment of the invention the oral disc comprises glidant. Silicon dioxide may be used as a glidant. Other glidants usable for the oral disc may also be used within the scope of the invention.

In an embodiment of the invention the oral disc comprises lubricant. Magnesium stearate and/or sodium stearyl fumarate may be used as a lubricant. Other lubricants usable for the oral disc may also be used within the scope of the invention.

According to the invention, the one or more cannabinoids may be selected from various cannabinoids.

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which may have high affinity ligands of CB1 and CB2 receptors.

“Phytocannabinoids” are cannabinoids that originate in nature and can be found in the cannabis plant. The phytocannabinoids can be present in an extract including a botanical drug substance, isolated, or reproduced synthetically.

“Syntho-cannabinoids” are those compounds capable of interacting with the cannabinoid receptors (CB1 and/or CB2) but are not found endogenously or in the cannabis plant. Examples include WIN 55212 and rimonabant.

An “isolated phytocannabinoid” is one which has been extracted from the cannabis plant and purified to such an extent that the additional components such as secondary and minor cannabinoids and the non-cannabinoid fraction have been substantially removed.

A “synthetic cannabinoid” is one which has been produced by chemical synthesis. This term includes modifying an isolated phytocannabinoid, by, for example, forming a pharmaceutically acceptable salt thereof.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

A “highly purified” cannabinoid is defined as a cannabinoid that has been extracted from the cannabis plant and purified to the extent that other cannabinoids and non-cannabinoid components that are co-extracted with the cannabinoids have been substantially removed, such that the highly purified cannabinoid is greater than or equal to 95% (w/w) pure.

“Plant material” is defined as a plant or plant part (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Center for Drug Evaluation and Research.

In the context of this application the terms “cannabinoid extract” or “extract of cannabinoids”, which are used interchangeably, encompass “Botanical Drug Substances” derived from cannabis plant material. A Botanical Drug Substance is defined in the Guidance for Industry Botanical Drug Products Draft Guidance, August 2000, US Department of Health and Human Services, Food and Drug Administration Centre for Drug Evaluation and Research as: “A drug substance derived from one or more plants, algae, or macroscopic fungi. It is prepared from botanical raw materials by one or more of the following processes: pulverisation, decoction, expression, aqueous extraction, ethanolic extraction, or other similar processes.” A botanical drug substance does not include a highly purified or chemically modified substance derived from natural sources. Thus, in the case of cannabis, “botanical drug substances” derived from cannabis plants do not include highly purified, Pharmacopoeial grade cannabinoids.

The term “Cannabis plant(s)” encompasses wild type Cannabis sativa and also variants thereof, including cannabis chemovars which naturally contain different amounts of the individual cannabinoids, Cannabis sativa subspecies indica including the variants var. indica and var. kafiristanica, Cannabis indica, Cannabis ruderalis and also plants which are the result of genetic crosses, self-crosses or hybrids thereof. The term “Cannabis plant material” is to be interpreted accordingly as encompassing plant material derived from one or more cannabis plants. For the avoidance of doubt it is hereby stated that “cannabis plant material” includes dried cannabis biomass.

Preferably the one or more cannabinoids are selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC. This list is not exhaustive and merely details the cannabinoids which are identified in the present application for reference.

So far, more than 120 different phytocannabinoids have been identified which are within the scope of the present invention.

Cannabinoids can be split into different groups as follows: Phytocannabinoids; Endocannabinoids; and Synthetic cannabinoids.

Cannabinoid receptors can be activated by three major groups of agonist ligands, for the purposes of the present invention and whether or not explicitly denominated as such herein, lipophilic in nature and classed respectively as: endocannabinoids (produced endogenously by mammalian cells); phytocannabinoids (such as cannabidiol, produced by the cannabis plant); and, synthetic cannabinoids (such as HU-210).

Phytocannabinoids can be found as either the neutral carboxylic acid form or the decarboxylated form depending on the method used to extract the cannabinoids. For example, it is known that heating the carboxylic acid form will cause most of the carboxylic acid form to decarboxylate.

Phytocannabinoids can also occur as either the pentyl (5 carbon atoms) or propyl (3 carbon atoms) variant. For example, the phytocannabinoid THC is known to be a CB1 receptor agonist whereas the propyl variant THCV has been discovered to be a CB1 receptor antagonist meaning that it has almost opposite effects.

According to the invention, examples of phytocannabinoids may be cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC.

The formulation according to the present invention may also comprise at least one cannabinoid selected from those disclosed in A. Douglas Kinghorn et al., Phytocannabinoids, Vol. 103, Chapter 1, pages 1-30.

Examples of endocannabinoids are molecules that activate the cannabinoid receptors within the body. Examples include 2-arachidonyl glycerol (2AG), 2-arachidonyl glyceryl ether (2AGE), arachidonyl dopamine, and arachidonyl ethanolamide (anandamide). Structurally related endogenous molecules have been identified that share similar structural features, but that display weak or no activity towards the cannabinoid receptors but are also termed endocannabinoids. Examples of these endocannabinoid lipids include 2-acyl glycerols, alkyl or alkenyl glyceryl ethers, acyl dopamines and N-acylethanolamides that contain alternative fatty acid or alcohol moieties, as well as other fatty acid amides containing different head groups. These include N-acylserines as well as many other N-acylated amino acids. Examples of cannabinoid receptor agonists are neuromodulatory and affect short-term memory, appetite, stress response, anxiety, immune function and analgesia.

In one embodiment the cannabinoid is palmitoylethanolamide (PEA) which is an endogenous fatty acid amide belonging to the class of nuclear factor agonists.

Synthetic cannabinoids encompass a variety of distinct chemical classes: the cannabinoids structurally related to THC, the cannabinoids not related to THC, such as (cannabimimetics) including the aminoalkylindoles, 1,5-diarylpyrazoles, quinolines, and arylsulfonamides, and eicosanoids related to the endocannabinoids. All or any of these cannabinoids can be used in the present invention.

It is preferred that the formulation comprises one or two primary cannabinoids, which are preferably selected from the group consisting of, cannabidiol (CBD) or cannabidivarin (CBDV), tetrahydrocannabinol (THC), tetrahydrocannabivarin (THCV), tetrahydrocannabinolic acid (THCA), cannabigerol (CBG) and cannabidiolic acid (CBDA) or a combination thereof. It is preferred that the formulation comprises cannabidiol and/or tetrahydrocannabinol.

Preferably, the oral disc of the present invention may be used for the treatment or alleviation of pain, epilepsy, cancer, nausea, inflammation, congenital disorders, neurological disorders, oral infections, dental pain, sleep apnea, psychiatric disorders, gastrointestinal disorders, inflammatory bowel disease, appetite loss, diabetes and fibromyalgia.

In a further aspect of the present invention the oral cannabinoid formulation is suitable for use in the treatment of conditions requiring the administration of a neuroprotectant or anti-convulsive medication.

The oral cannabinoid formulation may be for use in the treatment of seizures.

The oral cannabinoid formulation may be for use in the treatment of Dravet syndrome, Lennox Gastaut syndrome, myoclonic seizures, juvenile myoclonic epilepsy, refractory epilepsy, schizophrenia, juvenile spasms, West syndrome, infantile spasms, refractory infantile spasms, tuberous sclerosis complex, brain tumours, neuropathic pain, cannabis use disorder, post-traumatic stress disorder, anxiety, early psychosis, Alzheimer's disease, and autism.

The following non-limiting examples illustrate different variations of the present invention. The examples are meant for indicating the inventive concept; hence the mentioned examples should not be understood as exhaustive for the present. In particular, CBD is used as an exemplary compound, but may also be another cannabinoid.

### EXAMPLES

### Example 1: Preparation of Oral Discs Comprising a First and a Second Layer

The composition of the second layer, i.e., the non-mucoadhesive layer, is prepared with sugar alcohol unless specifically otherwise noted. The composition is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of first layer, i.e., the mucoadhesive contacting layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hopper of a tableting machine.

The second layer is then compressed at a compression force of about 1-5 kN, after which the first layer is fused by compression to the second layer at a compression force of about 8-15 kN. Punch used unless otherwise specified: 8.00 mm, circular, convex dimple, D tooling.

In an alternative process configuration, the second layer is compressed at a compression force of about 3-8 kN, after which the first layer is fused by compression to the second layer at a compression force of about 20-30 kN. Punch used unless otherwise specified: 12.00 mm, circular, shallow concave, B tooling.

The oral discs are manufactured on a lab scale machine, for example RIVA Piccola tablet press.

The tablet machine is commissioned by adjusting the fill depth and compression force so the weight and hardness of oral discs match the acceptance criteria. A pre-compression force could be included to avoid capping.

### Example 1A: Preparation of Oral Discs Configured without Layers

The oral discs are prepared with sugar alcohol unless specifically otherwise noted. The composition of the oral discs is prepared by pouring about half the sugar alcohol into a mixing bowl, followed by the other ingredients except lubricant, into a mixing bowl, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The lubricated powder blend is sequentially transferred to the hopper of a tableting machine.

The oral disc is then compressed at a compression force of about 20-30 kN. Punch used unless otherwise specified: 12.00 mm, circular, shallow concave, B tooling.

The oral discs are manufactured on a lab scale machine, for example RIVA Piccola tablet press. The tablet machine is commissioned by adjusting the fill depth and compression force so the weight and hardness of discs match the acceptance criteria. A pre-compression force could be included to avoid capping.

### Example 1B: Preparation of 3-Layer Oral Discs

The composition of the third layer is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The composition of the second layer, i.e., the non-mucoadhesive layer, is prepared with sugar alcohol unless specifically otherwise noted. The composition of the second layer is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of the first layer, i.e. the mucoadhesive contacting layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hoppers of a tableting machine.

The first layer is compressed at a compression force of about 1-6 kN, after which the second layer is fused by compression to the first layer at a compression force of about 2-8 kN. Then, the third layer is fused by compression to the second layer at a compression force of about 20-40 kN. Punch used unless otherwise specified: 12.00 mm, circular, shallow concave, B tooling.

The oral discs are manufactured on a PZ-TRE rotary tablet press available from B&D Italia, but other standard equipment for making tri-layer tablets may be used as well, such as a PTK PR3500 or a Hata tri-layer tablet press. The tablet machine is commissioned by adjusting the fill depth and compression force so the weight and hardness of tablets match the acceptance criteria. A pre-compression force could be included to avoid capping.

### Example 2A: Cannabinoid Sources

150 mg oral discs were made each with 85 mg second layer and 65 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 1
Compositions of first and second layers.

| | C1 | C2 | C3 | C4 | C5 | C6 | C7 | C8 | C9 |
|---|---|---|---|---|---|---|---|---|---|
| Cannabinoid dose | 10 | 10 | 10 | 10 | 10 | 30 | 30 | 30 | 30 |
| 1^{st} layer [mg] | | | | | | | | | |
| Raw material | Content in weight percent of 1^{st} layer | | | | | | | | |
| 1^{st} layer | | | | | | | | | |
| Gum arabic | 60 | 60 | 60 | 60 | 60 | 40 | 40 | 40 | 40 |
| Mannitol | 24 | 24 | 24 | 24 | 24 | 13 | 13 | 13 | 13 |
| MgSt | 1 | 1 | 1 | 1 | 1 | 1 | 1 | 1 | 1 |
| CBD | 15 | — | — | | 15 | 46 | — | — | — |
| CBG | — | 15 | — | — | — | — | 46 | — | — |
| CBN | — | — | 15 | — | — | — | — | 46 | — |
| THC | — | — | — | 15 | — | — | — | — | 46 |
| Total 1st layer | 100 | 100 | 100 | 100 | 100 | 100 | 100 | 100 | 100 |
| Cannabinoid dose | — | — | — | — | 20 | — | — | — | — |
| 2^{nd} layer [mg] | | | | | | | | | |
| Raw material | Content in weight percent of 2^{nd} layer | | | | | | | | |
| 2^{nd} layer | | | | | | | | | |
| HPC | 7.5 | 7.5 | 7.5 | 7.5 | 7.5 | 7.5 | 7.5 | 7.5 | 7.5 |
| Mannitol | 90.2 | 90.2 | 90.2 | 90.2 | 66.2 | 90.2 | 90.2 | 90.2 | 90.2 |
| HIS | 0.3 | 0.3 | 0.3 | 0.3 | 0.3 | 0.3 | 0.3 | 0.3 | 0.3 |
| MgSt | 2 | 2 | 2 | 2 | 2 | 2 | 2 | 2 | 2 |
| CBD | — | — | — | — | 24 | — | — | — | — |
| Total 2^{nd} layer | 100 | 100 | 100 | 100 | 100 | 100 | 100 | 100 | 100 |

HPC = hydroxypropyl cellulose.
HIS = high intensity sweetener.

For all the samples, the cannabinoids were applied by means of a premix of the cannabinoids and mannitol.

Preferred high intensity sweeteners (HIS) may e.g. be sucralose, acesulfame potassium, and mixtures thereof.

In the above MgSt (magnesium stearate) is used as lubricant. Other lubricants, such as sodium stearyl fumerate may also be usable within the scope of the invention.

### Example 2B: Binder Variations

150 mg oral discs were made each with 85 mg second layer and 65 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 2
Compositions of first and second layers.

| | C10 | C11 | C12 | C13 | C14 | C15 | C16 | C17 |
|---|---|---|---|---|---|---|---|---|
| Cannabinoid | 15 | 15 | 15 | 15 | 15 | 15 | 15 | 15 |
| dose 1^{st} layer | | | | | | | | |
| [mg] | | | | | | | | |
| Raw material | Content in weight percent of 1^{st} layer | | | | | | | |
| 1^{st} layer | | | | | | | | |
| Gum arabic | 60 | 60 | 60 | 60 | 60 | 60 | 60 | 60 |
| Mannitol | 11 | 6 | 8.5 | 8.5 | 1 | 6 | 11 | 6 |
| MgSt | 1 | 1 | 1 | 1 | 1 | 1 | 1 | 1 |
| HPC | 5 | 10 | — | — | 7.5 | 5 | — | — |
| MCC | — | — | 7.5 | — | 7.5 | — | — | — |
| CMC | — | — | — | 7.5 | — | — | — | — |
| HPMC | — | — | — | — | — | 5 | 5 | 10 |
| CBD | 23 | 23 | 23 | 23 | 23 | 23 | 23 | 23 |
| Total 1st layer | 100 | 100 | 100 | 100 | 100 | 100 | 100 | 100 |
| Raw material | Content in weight percent of 2^{nd} layer | | | | | | | |
| 2^{nd} layer | | | | | | | | |
| MgSt | 2 | 2 | 2 | 2 | 2 | 2 | 2 | 2 |
| Mannitol | 92.4 | 87.4 | 89.9 | 89.9 | 82.4 | 87.4 | 92.4 | 87.4 |
| HPC | 5 | 10 | — | — | 7.5 | 5 | — | — |
| MCC | — | — | 7.5 | — | 7.5 | — | — | — |
| CMC | — | — | — | 7.5 | — | — | — | — |
| HPMC | — | — | — | — | — | 5 | 5 | 10 |
| HIS | 0.6 | 0.6 | 0.6 | 0.6 | 0.6 | 0.6 | 0.6 | 0.6 |
| Total 2nd layer | 100 | 100 | 100 | 100 | 100 | 100 | 100 | 100 |

HPC = hydroxypropyl cellulose.
HIS = high intensity sweetener.
MCC = microcrystalline cellulose.
CMC = carboxymethyl cellulose.
HPMC = hydroxypropyl methylcellulose.

### Example 2C: Binder Variations

150 mg oral discs were made each with 85 mg second layer and 65 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 3
Compositions of first and second layers.

| | C18 | C19 | C20 | C21 | C22 | C23 | C24 | C25 |
|---|---|---|---|---|---|---|---|---|
| Cannabinoid | 15 | 15 | 15 | 15 | 15 | 15 | 15 | 15 |
| dose 1^{st} layer | | | | | | | | |
| [mg] | | | | | | | | |
| Raw material | Content in weight percent of 1^{st} layer | | | | | | | |
| 1^{st} layer | | | | | | | | |
| Gum arabic | 60 | 60 | 60 | 60 | 60 | 60 | 60 | 60 |
| Mannitol | 16 | 16 | 16 | 16 | 8.5 | 8.5 | 8.5 | 8.5 |
| MgSt | 1 | 1 | 1 | 1 | 1 | 1 | 1 | 1 |
| HPC | — | — | — | — | 7.5 | — | — | — |
| MCC | — | — | — | — | — | 7.5 | — | — |
| CMC | — | — | — | — | — | — | 7.5 | — |
| HPMC | — | — | — | — | — | — | — | 7.5 |
| CBD | 23 | 23 | 23 | 23 | 23 | 23 | 23 | 23 |
| Total 1st layer | 100 | 100 | 100 | 100 | 100 | 100 | 100 | 100 |
| Raw material | Content in weight percent of 2^{nd} layer | | | | | | | |
| 2^{nd} layer | (15 mg cannabinoids applied in all examples) | | | | | | | |
| MgSt | 2 | 2 | 2 | 2 | 2 | 2 | 2 | 2 |
| Mannitol | 74.4 | 69.4 | 74.4 | 69.4 | 74.4 | 69.4 | 74.4 | 69.4 |
| HPC | 5 | — | 5 | — | 5 | — | 5 | — |
| HPMC | — | 10 | — | 10 | — | 10 | — | 10 |
| HIS | 0.6 | 0.6 | 0.6 | 0.6 | 0.6 | 0.6 | 0.6 | 0.6 |
| CBD | 18 | 18 | 18 | 18 | 18 | 18 | 18 | 18 |
| Total 2nd layer | 100 | 100 | 100 | 100 | 100 | 100 | 100 | 100 |

HPC = hydroxypropyl cellulose.
HIS = high intensity sweetener.
HPMC = hydroxypropyl methylcellulose.

### Example 2D: Sugar Alcohol Variations

300 mg oral discs were made each with 200 mg second layer and 100 mg first layer. The oral discs were prepared according to Example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 4
Compositions of first and second layers.
HPC = hydroxypropyl cellulose.

| | C26 | C27 | C28 | C29 | C30 |
|---|---|---|---|---|---|
| Cannabinoid dose | 15 | 15 | 15 | 15 | 15 |
| 1^{st} layer [mg] | | | | | |
| Raw material | Content in weight percent of 1^{st} layer | | | | |
| 1^{st} layer | | | | | |
| Gum arabic | 60 | 60 | 60 | 60 | 60 |
| Xylitol | 24 | — | — | — | — |
| Isomalt | — | 24 | — | — | — |
| Maltitol | — | — | 24 | — | — |
| Erythritol | — | — | — | 12 | — |
| Sorbitol | — | — | — | 12 | 24 |
| MgSt | 1 | 1 | 1 | 1 | 1 |
| CBD | 15 | 15 | 15 | 15 | 15 |
| Total 1^{st} layer | 100 | 100 | 100 | 100 | 100 |
| Raw material | Content in weight percent of 2^{nd} layer | | | | |
| 2^{nd} layer | | | | | |
| Mannitol | 91.5 | — | — | — | 46.0 |
| Isomalt | — | 91.5 | — | — | — |
| Maltitol | — | — | 91.5 | — | — |
| Erythritol | — | — | — | 91.5 | — |
| Sorbitol | — | — | — | — | 45.5 |
| HPC | 7.5 | 7.5 | 7.5 | 7.5 | 7.5 |
| MgSt | 1 | 1 | 1 | 1 | 1 |
| Total 2^{nd} layer | 100 | 100 | 100 | 100 | 100 |

### Example 2E: Mucoadhesive Variations

150 mg oral discs were made each with 75 mg second layer and 75 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 4A
Compositions of first and second layers.

| | C31 | C32 | C33 | C34 | C35 | C36 | C37 |
|---|---|---|---|---|---|---|---|
| Cannabinoid | 15 | 7.5 | 3.75 | 15 | 15 | 15 | 15 |
| dose 1^{st} layer | | | | | | | |
| [mg] | | | | | | | |
| Raw material | Content in weight percent of 1^{st} layer | | | | | | |
| 1^{st} layer | | | | | | | |
| Gum arabic | 50 | 70 | 90 | — | 60 | 40 | 20 |
| Na-alginate | — | — | — | 60 | — | — | — |
| Mannitol | 29 | 19 | 4 | 19 | 18.8 | 38.8 | 59 |
| MgSt | 1 | 1 | 1 | 1 | 1 | 1 | 1 |
| HIS | — | — | — | — | 0.2 | 0.2 | — |
| CBD | 20 | 10 | 5 | 20 | 20 | 20 | 20 |
| Total 1^{st} layer | 100 | 100 | 100 | 100 | 100 | 100 | 100 |
| Raw material | Content in weight percent of 2^{nd} layer | | | | | | |
| 2^{nd} layer | | | | | | | |
| Gum arabic | 2 | 2 | 2 | — | — | — | — |
| Xanthan gum | — | — | — | 2 | 2 | 2 | 2 |
| Mannitol | 87.9 | 87.9 | 87.9 | 87.9 | 87.9 | 87.9 | 87.9 |
| HIS | 0.6 | 0.6 | 0.6 | 0.6 | 0.6 | 0.6 | 0.6 |
| MgSt | 2 | 2 | 2 | 2 | 2 | 2 | 2 |
| HPC | 7.5 | 7.5 | 7.5 | 7.5 | 7.5 | 7.5 | 7.5 |
| Total 2^{nd} layer | 100 | 100 | 100 | 100 | 100 | 100 | 100 |

HPC = hydroxypropyl cellulose.
HIS = high intensity sweetener.

### Example 2F: Binder Variations

150 mg oral discs were made each with 85 mg second layer and 65 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 5
Compositions of first and second layers.

| | CA10 | CA11 | CA12 | CA13 | CA14 | CA15 | CA16 | CA17 |
|---|---|---|---|---|---|---|---|---|
| Raw material | Content in weight percent of 1^{st} layer | | | | | | | |
| 1^{st} layer | | | | | | | | |
| Gum arabic | 60 | 60 | 60 | 60 | 60 | 60 | 60 | 60 |
| Mannitol | 34 | 29 | 31.5 | 31.5 | 24 | 29 | 34 | 29 |
| MgSt | 1 | 1 | 1 | 1 | 1 | 1 | 1 | 1 |
| HPC | 5 | 10 | — | — | 7.5 | 5 | — | — |
| MCC | — | — | 7.5 | — | 7.5 | — | — | — |
| CMC | — | — | — | 7.5 | — | — | — | — |
| HPMC | — | — | — | — | — | 5 | 5 | 10 |
| Total 1st layer | 100 | 100 | 100 | 100 | 100 | 100 | 100 | 100 |
| Cannabinoid | 25 | 25 | 25 | 25 | 25 | 25 | 25 | 25 |
| dose 2^{nd} layer | | | | | | | | |
| [mg] | | | | | | | | |
| Raw material | Content in weight percent of 2^{nd} layer | | | | | | | |
| 2^{nd} layer | | | | | | | | |
| MgSt | 2 | 2 | 2 | 2 | 2 | 2 | 2 | 2 |
| Mannitol | 63.0 | 58.0 | 60.5 | 60.5 | 53.0 | 58.0 | 63.0 | 58.0 |
| HPC | 5 | 10 | — | — | 7.5 | 5 | — | — |
| MCC | — | — | 7.5 | - | 7.5 | — | — | — |
| CMC | — | — | — | 7.5 | — | — | — | — |
| HPMC | — | — | — | — | — | 5 | 5 | 10 |
| HIS | 0.6 | 0.6 | 0.6 | 0.6 | 0.6 | 0.6 | 0.6 | 0.6 |
| CBD | 29.4 | 29.4 | 29.4 | 29.4 | 29.4 | 29.4 | 29.4 | 29.4 |
| Total 2nd layer | 100 | 100 | 100 | 100 | 100 | 100 | 100 | 100 |

HPC = hydroxypropyl cellulose.
HIS = high intensity sweetener.
MCC = microcrystalline cellulose.
CMC = carboxymethyl cellulose.
HPMC = hydroxypropyl methylcellulose.

### Example 2H: Addition of Fillers

400 mg oral discs were made each with 300 mg second layer and 100 mg first layer. The oral discs were prepared according to Example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 5A
Compositions of first and second layers.

| | CA37 | CA38 | CA39 | CA40 | CA41 | CA42 |
|---|---|---|---|---|---|---|
| Raw material | Content in weight percent of 1st module | | | | | |
| 1^{st} layer | | | | | | |
| Sorbitol | 28 | 28 | 28 | 50 | 28 | 6 |
| Calcium | 6 | 6 | 6 | 4 | 6 | 8 |
| carbonate | | | | | | |
| HPC | 5 | 5 | 5 | 5 | 5 | 5 |
| Gum arabic | 60 | 60 | 60 | 40 | 60 | 80 |
| MgSt | 1 | 1 | 1 | 1 | 1 | 1 |
| Total 1^{st} layer | 100 | 100 | 100 | 100 | 100 | 100 |
| Raw material | Content in weight percent of 2^{nd} module | | | | | |
| 2^{nd} layer | | | | | | |
| CDB dose 2^{nd} | 40 | 40 | 40 | 40 | 40 | 40 |
| layer [mg] | | | | | | |
| Xylitol | 85 | 80 | 75 | 75 | 75 | 75 |
| HIS | 0.2 | 0.2 | 0.2 | 0.2 | 0.2 | 0.2 |
| CBD | 13.3 | 13.3 | 13.3 | 13.3 | 13.3 | 13.3 |
| HPC | — | 5 | 10 | 10 | 10 | 10 |
| Flavor | 0.5 | 0.5 | 0.5 | 0.5 | 0.5 | 0.5 |
| MgSt | 1 | 1 | 1 | 1 | 1 | 1 |
| Total 2^{nd} layer | 100.0 | 100.0 | 100.0 | 100.0 | 100.0 | 100.0 |

HPC = hydroxypropyl cellulose.
HIS = high intensity sweetener.

In Table 5A, sucralose was used as high intensity sweetener. Flavor may e.g. be combination of peppermint and menthol.

### Example 2I

300 mg one-layer oral discs. The oral discs were prepared according to Example 1A.

Punch used: 12.00 mm, circular, shallow concave, B tooling.

The oral disc is compressed at a compression force of about 20-40 kN.

TABLE 5B
Compositions of first and second layers. HPC = hydroxypropyl
cellulose. HIS = high intensity sweetener.

| | CB37 | CB38 | CB39 |
|---|---|---|---|
| CBD dose tablet [mg] | 10 | 10 | 10 |
| Raw material | Content in weight percent of 1st module | | |
| Xylitol | 34.4 | 29.4 | 24.4 |
| CBD | 3.3 | 3.3 | 3.3 |
| HIS | 0.2 | 0.2 | 0.2 |
| Gum arabic | 60 | 60 | 60 |
| HPC | — | 5 | 10 |
| Flavor | 1.1 | 1.1 | 1.1 |
| MgSt | 1 | 1 | 1 |
| Total | 100 | 100 | 100 |

In Table 5B, sucralose was used as high intensity sweetener. Flavor may e.g. be combination of peppermint and menthol.

### Example 2J

270 mg oral discs were made each with 70 mg first layer and 200 mg second layer. The oral discs were prepared according to Example 1.

Punch used: 8.00 mm, circular, convex dimple, D tooling.

The second layer is compressed at a compression force of about 3-8 kN, after which the first layer is fused by compression to the second layer at a compression force of about 20-30 kN.

TABLE 5C
Compositions of first and second layers. HPC = hydroxypropyl
cellulose. HIS = high intensity sweetener.

| | CC37 | CC38 | CC39 |
|---|---|---|---|
| Raw material 1^{st} layer | Content in weight percent of 1st module | | |
| Sorbitol | 24.8 | 24.8 | 24.8 |
| CaCO3 | 6 | 6 | 6 |
| HIS | 0.2 | 0.2 | 0.2 |
| HPC | 5 | 5 | 5 |
| Gum arabic | 60 | 60 | 60 |
| Flavor | 3 | 3 | 3 |
| MgSt | 1 | 1 | 1 |
| Total 1^{st} layer | 100 | 100 | 100 |
| CBD dose 2^{nd} layer [mg] | 5 | 5 | 5 |
| Raw material 2^{nd} layer | Content in weight percent of 2^{nd} module | | |
| Mannitol | — | 86.8 | — |
| Xylitol | 89.8 | — | 85.8 |
| CBD | 2.5 | 2.5 | 2.5 |
| Xanthan gum | — | 6 | 3 |
| Sodium Alginate | — | 2 | 1 |
| HIS | 0.2 | 0.2 | 0.2 |
| HPC | 5 | — | 5 |
| Flavor | 1.5 | 1.5 | 1.5 |
| MgSt | 1 | 1 | 1 |
| Total 2^{nd} layer | 100 | 100 | 100 |

In table 5C, sucralose was used as high intensity sweetener. Flavor may e.g. be combination of peppermint and menthol.

### Example 2K

Oral discs with varying size were prepared according to Example 1B.

Punch used for samples CD37-CD42:12.00 mm, circular, shallow concave, B tooling.

Punch used for sample CD43:10.00 mm, circular, convex dimple.

The first layer is compressed at a compression force of about 1-6 kN, after which the second layer is fused by compression to the first layer at a compression force of about 2-8 kN. Then, the third layer is fused by compression to the second layer at a compression force of about 20-40 kN.

TABLE 5D
Compositions of first and second layers.

| | CD37 | CD38 | CD39 | CD40 | CD41 | CD42 | CD43 |
|---|---|---|---|---|---|---|---|
| 1st layer | 100 | 100 | 100 | 100 | 100 | 100 | 75 |
| weight [mg] | | | | | | | |
| Raw material | Content in weight percent of 1st module | | | | | | |
| 1^{st} layer | | | | | | | |
| CaCO3 | 10 | 10 | 10 | 10 | 10 | 10 | 10 |
| HPC | 5 | 5 | 5 | 5 | 5 | 5 | 5 |
| Gum arabic | 84 | 84 | 84 | 84 | 84 | 84 | 84 |
| MgSt | 1 | 1 | 1 | 1 | 1 | 1 | 1 |
| Total 1^{st} layer | 100 | 100 | 100 | 100 | 100 | 100 | 100 |
| 2nd layer | 300 | 300 | 300 | 300 | 300 | 300 | 100 |
| weight [mg] | | | | | | | |
| CBD dose 2^{nd} | 20 | 20 | 20 | 20 | 20 | 20 | 20 |
| layer [mg] | | | | | | | |
| Raw material | Content in weight percent of 2nd module | | | | | | |
| 2nd layer | | | | | | | |
| Xylitol | 81.6 | 81.6 | 73.6 | 73.6 | 77.6 | 77.6 | 68.3 |
| Xanthan gum | — | — | 6 | 6 | 3 | 3 | — |
| Sodium alginate | — | — | 2 | 2 | 1 | 1 | — |
| CBD | 6.7 | 6.7 | 6.7 | 6.7 | 6.7 | 6.7 | 20.0 |
| HIS | 0.2 | 0.2 | 0.2 | 0.2 | 0.2 | 0.2 | 0.2 |
| HPC | 10 | 10 | 10 | 10 | 10 | 10 | 10 |
| Flavor | 1.5 | 1.5 | 1.5 | 1.5 | 1.5 | 1.5 | 1.5 |
| Total 2^{nd} layer | 100 | 100 | 100 | 100 | 100 | 100 | 100 |
| 3^{rd} layer | 100 | 100 | 100 | 100 | 100 | 100 | 50 |
| weight [mg] | | | | | | | |
| CBD dose 3^{rd} | 20 | 20 | 20 | 20 | 20 | 20 | 20 |
| layer [mg] | | | | | | | |
| Raw material | Content in weight percent of 3^{rd} module | | | | | | |
| 3^{rd} layer | | | | | | | |
| Pearlitol flash | 77.4 | 37.4 | 77.4 | 37.4 | 39.4 | 77.4 | 57.4 |
| Xylitol | — | 40 | — | 40 | 30 | — | — |
| Xanthan gum | — | — | — | — | 6 | — | — |
| Sodium alginate | — | — | — | — | 2 | — | — |
| CBD | 20 | 20 | 20 | 20 | 20 | 20 | 40 |
| HIS | 0.1 | 0.1 | 0.1 | 0.1 | 0.1 | 0.1 | 0.1 |
| HPC | 1 | 1 | 1 | 1 | 1 | 1 | 1 |
| Flavor | 0.5 | 0.5 | 0.5 | 0.5 | 0.5 | 0.5 | 0.5 |
| MgSt | 1 | 1 | 1 | 1 | 1 | 1 | 1 |
| Total 3^{rd} layer | 100 | 100 | 100 | 100 | 100 | 100 | 100 |

HPC = hydroxypropyl cellulose.
HIS = high intensity sweetener.

In Table 5D, sucralose was used as high intensity sweetener. Flavor may e.g. be combination of peppermint and menthol.

It is noted that Pearlitol flash is a commercially available ready to use system comprising mannitol and starch in a mannitol to starch weight ratio of approximately 4:1.

### Example 2L

500 mg oral discs were made each with 100 mg first layer, 300 mg second layer and 100 mg third layer. The oral discs were prepared according to Example 1B.

Punch used: 12.00 mm, circular, shallow concave, B tooling.

The first layer is compressed at a compression force of about 1-6 kN, after which the second layer is fused by compression to the first layer at a compression force of about 2-8 kN. Then, the third layer is fused by compression to the second layer at a compression force of about 20-40 kN.

TABLE 5E
Compositions of first and second layers.

| | CE37 | CE38 | CE39 | CE40 | CE41 | CE42 |
|---|---|---|---|---|---|---|
| CBD dose 1^{st} | — | 2.5 | — | — | 5 | — |
| layer [mg] | | | | | | |
| Raw material | Content in weight percent of 1st module | | | | | |
| 1^{st} layer | | | | | | |
| CaCO3 | 10 | 7.5 | 10 | 10 | 5 | 10 |
| HPC | 5 | 5 | 5 | 5 | 5 | 5 |
| Gum arabic | 84 | 84 | 84 | 84 | 84 | 84 |
| CBD | — | 2.5 | — | — | 5 | — |
| MgSt | 1 | 1 | 1 | 1 | 1 | 1 |
| Total 1^{st} layer | 100 | 100 | 100 | 100 | 100.0 | 100.0 |
| CBD dose 2^{nd} | 5 | 2.5 | 5 | 30 | 60 | 30 |
| layer [mg] | | | | | | |
| THC dose 2^{nd} | — | — | — | — | — | 10 |
| layer [mg] | | | | | | |
| Raw material | Content in weight percent of 2^{nd} module | | | | | |
| 2^{nd} layer | | | | | | |
| Xylitol | 86.63 | 87.47 | 86.63 | 78.3 | 68.3 | 75 |
| CBD | 1.67 | 0.83 | 1.67 | 10 | 20 | 10 |
| THC | — | — | — | — | — | 3.3 |
| HIS | 0.2 | 0.2 | 0.2 | 0.2 | 0.2 | 0.2 |
| HPC | 10 | 10 | 10 | 10 | 10 | 10 |
| Flavor | 1.5 | 1.5 | 1.5 | 1.5 | 1.5 | 1.5 |
| Total 2^{nd} layer | 100.0 | 100.0 | 100.0 | 100.0 | 100.0 | 100.0 |
| CBD dose 3^{rd} | 5 | 5 | — | — | 10 | 20 |
| layer [mg] | | | | | | |
| THC dose 3^{rd} | — | — | — | — | — | 10 |
| layer [mg] | | | | | | |
| Raw material | Content in weight percent of 3^{rd} module | | | | | |
| 3^{rd} layer | | | | | | |
| Pearlitol flash | 92.4 | 92.4 | 97.4 | 97.4 | 87.4 | 47.4 |
| Xylitol | — | — | — | — | — | 20 |
| THC | — | — | — | — | — | 10 |
| CBD | 5 | 5 | — | — | 10 | 20 |
| HIS | 0.1 | 0.1 | 0.1 | 0.1 | 0.1 | 0.1 |
| HPC | 1 | 1 | 1 | 1 | 1 | 1 |
| Flavor | 0.5 | 0.5 | 0.5 | 0.5 | 0.5 | 0.5 |
| MgSt | 1 | 1 | 1 | 1 | 1 | 1 |
| Total 3^{rd} layer | 100 | 100 | 100 | 100 | 100 | 100 |

HPC = hydroxypropyl cellulose.
HIS = high intensity sweetener.

In table 5E, sucralose was used as high intensity sweetener. Flavor may e.g. be combination of peppermint and menthol.

### Example 2M

500 mg oral discs were made each with 100 mg first layer. 300 mg second layer and 100 mg third layer. The oral discs were prepared according to Example 1B.

Punch used: 12.00 mm, circular, shallow concave. B tooling.

The first layer is compressed at a compression force of about 1-6 kN, after which the second layer is fused by compression to the first layer at a compression force of about 2-8 kN. Then, the third layer is fused by compression to the second layer at a compression force of about 20-40 kN.

TABLE 5F
Compositions of first and second layers.

| | CF37 | CF38 | CF39 | CF40 | CF41 | CF42 |
|---|---|---|---|---|---|---|
| CBD dose 1^{st} layer [mg] | 20 | — | 20 | — | 20 | — |
| THC dose 1^{st} layer [mg] | — | 20 | — | 20 | — | 20 |
| Raw material 1^{st} layer | Content in weight percent of 1st module | | | | | |
| CaCO3 | 7.8 | 7.8 | 7.8 | 7.8 | 7.8 | 7.8 |
| HPC | 5 | 5 | 5 | 5 | 5 | 5 |
| Gum arabic | 66.2 | 66.2 | 66.2 | 66.2 | 66.2 | 66.2 |
| CBD | 20 | — | 20 | — | 20 | — |
| THC | — | 20 | — | 20 | — | 20 |
| MgSt | 1 | 1 | 1 | 1 | 1 | 1 |
| Total 1^{st} layer | 100 | 100 | 100 | 100 | 100 | 100 |
| CBD dose 2^{nd} layer [mg] | 40 | — | — | — | 40 | — |
| THC dose 2^{nd} layer [mg] | — | 40 | — | — | — | 40 |
| Raw material 2^{nd} layer | Content in weight percent of 2^{nd} module | | | | | |
| Xylitol | 75 | 75 | 88.3 | 88.3 | 75 | 75 |
| CBD | 13.3 | — | — | — | 13.3 | — |
| THC | — | 13.3 | — | — | — | 13.3 |
| HIS | 0.2 | 0.2 | 0.2 | 0.2 | 0.2 | 0.2 |
| HPC | 10 | 10 | 10 | 10 | 10 | 10 |
| Flavor | 1.5 | 1.5 | 1.5 | 1.5 | 1.5 | 1.5 |
| Total 2^{nd} layer | 75 | 75 | 88.3 | 88.3 | 41.6 | 41.6 |
| CBD dose 3^{rd} layer [mg] | 40 | — | 20 | — | 15 | — |
| THC dose 3^{rd} layer [mg] | — | 40 | — | 20 | — | 15 |
| Raw material 3^{rd} layer | Content in weight percent of 3^{rd} module | | | | | |
| Pearlitol flash | 57.4 | 57.4 | 77.4 | 77.4 | 82.4 | 82.4 |
| CBD | 40 | — | 20 | — | 15 | — |
| THC | — | 40 | — | 20 | — | 15 |
| HIS | 0.1 | 0.1 | 0.1 | 0.1 | 0.1 | 0.1 |
| HPC | 1 | 1 | 1 | 1 | 1 | 1 |
| Flavor | 0.5 | 0.5 | 0.5 | 0.5 | 0.5 | 0.5 |
| MgSt | 1 | 1 | 1 | 1 | 1 | 1 |
| Total 3^{rd} layer | 100 | 100 | 100 | 100 | 100 | 100 |

HPC = hydroxypropyl cellulose.
HIS = high intensity sweetener.

In Table 5F, sucralose was used as high intensity sweetener. Flavor may e.g. be combination of peppermint and menthol.

### Example 2N

270 mg oral discs were made each with 70 mg first layer and 200 mg second layer. The oral discs were prepared according to Example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 5G
Compositions of first and second layers. HPC = hydroxypropyl
cellulose. HIS = high intensity sweetener.

| | CG37 | CG38 | CG39 |
|---|---|---|---|
| Raw material 1^{st} layer | Content in weight percent of 1st module | | |
| Sorbitol | 24.8 | 24.8 | — |
| Xylitol | — | — | 23.8 |
| Citric acid | — | — | 1 |
| CaCO3 | 6 | 6 | 6 |
| HIS | 0.2 | 0.2 | 0.2 |
| HPC | 5 | 5 | 5 |
| Gum arabic | 60 | 60 | 60 |
| Flavor | 3 | 3 | 3 |
| MgSt | 1 | 1 | 1 |
| Total 1^{st} layer | 100 | 100 | 100 |
| CBG dose 2^{nd} layer [mg] | 30 | — | 20 |
| CBN dose 2^{nd} layer [mg] | — | 20 | — |
| Raw material 2^{nd} layer | Content in weight percent of 2^{nd} module | | |
| Mannitol | 77.3 | 82.3 | — |
| Xylitol | — | — | 82.3 |
| CBG | 15 | — | 10 |
| CBN | — | 10 | — |
| HIS | 0.2 | 0.2 | 0.2 |
| HPC | 5 | 5 | 5 |
| Flavor | 1.5 | 1.5 | 1.5 |
| MgSt | 1 | 1 | 1 |
| Total 2nd layer | 100 | 100 | 100 |

In table 5G, sucralose was used as high intensity sweetener. Flavor may e.g. be combination of peppermint and menthol. Other organic acids such as malic acid may be used instead of citric acid.

### Example 2O

300 mg one-layer oral discs. The tablets were prepared according to Example 1A.

Punch used: 12.00 mm, circular, shallow concave, B tooling.

The oral disc is compressed at a compression force of about 20-40 kN.

TABLE 5H
Compositions of first and second layers. HPC = hydroxypropyl
cellulose. HIS = high intensity sweetener.

| | CH37 | CH38 |
|---|---|---|
| CBD dose tablet [mg] | 40 | 40 |
| Raw material | Content in weight | percent of 1st module |
| Xylitol | — | 27 |
| Mannitol | 27 | — |
| HPC | 3 | 3 |
| Gum arabic | 50 | 50 |
| Flavor + HIS | 0.7 | 0.7 |
| CaCO3 | 5 | 5 |
| CBD | 13.3 | 13.3 |
| MgSt | 1 | 1 |
| Total | 100 | 100 |

In Table 5H, sucralose was used as high intensity sweetener. Flavor may e.g. be peppermint.

### Example 2P

400 mg oral discs were made each with 300 mg second layer and 100 mg first layer. The oral discs were prepared according to Example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 51
Compositions of first and second layers. HPC = hydroxypropyl
cellulose. HIS = high intensity sweetener.

| | CI37 | CI38 | CI39 | CI40 |
|---|---|---|---|---|
| CBD dose 1^{st} layer [mg] | — | — | 20 | 20 |
| Raw material 1^{st} layer | Content in weight percent of 1st module | | | |
| Sorbitol | 27 | 27 | 25 | 25 |
| HPC | 5 | 5 | 5 | 5 |
| Gum arabic | 60 | 60 | 48 | 48 |
| CBD | — | — | 20 | 20 |
| CaCO3 | 6 | 6 | — | — |
| Flavor + HIS | 1 | 1 | 1 | 1 |
| MgSt | 1 | 1 | 1 | 1 |
| Total 1^{st} layer | 100 | 100 | 100 | 100 |
| CBD dose 2^{nd} layer [mg] | 40 | 40 | 20 | 20 |
| Raw material 2^{nd} layer | Content in weight percent of 2^{nd} module | | | |
| Xylitol | — | 82 | 88.5 | 81.5 |
| Mannitol | 82 | — | — | — |
| HPC | 3 | 3 | 3 | 10 |
| Flavor + HIS | 1.2 | 1.2 | 1.3 | 1.3 |
| CBD | 13.3 | 13.3 | 6.7 | 6.7 |
| MgSt | 0.5 | 0.5 | 0.5 | 0.5 |
| Total 2nd layer | 100 | 100 | 100 | 100 |

In Table 5I, sucralose was used as high intensity sweetener. Flavor may e.g. be peppermint.

### Example 3: Evaluation of Oral Discs

For evaluation a panel of 7 trained assessors was used. Each assessor repeated the evaluations twice.

The trained assessors abstain from eating and drinking at least 30 minutes before initiation of any test. Each trained assessor was a healthy person appointed on an objective basis according to specified requirements.

For testing the oral disc was weighted and placed in the mouth, between the upper lip and the gum, with the first layer, i.e. the mucoadhesive layer, facing the gum. At specific time points, e.g. 15, 30 and 60 minutes, the content of cannabinoids was measured in the remaining oral disc residue, if still present. Once the desired test time was achieved, the oral disc was taken out and weighed directly into a measuring glass to be used for analysis of cannabinoid content. The cannabinoid content was analyzed by means of standard HPLC technique after extraction into relevant buffer.

Optionally, the oral discs were evaluated with respect to mouth feel, taste and other sensory parameters during testing.

If dissolution of the oral disc is substantially complete before the specified time point was reached, the time of dissolution was registered as the in vivo dissolution time of the oral disc.

The evaluated oral discs were found to be highly suitable delivery vehicles for cannabinoids, giving desirable mouthfeel and taste profile while providing a surprisingly sustained release of cannabinoids.

### Example 4: Variation in Sugar Alcohols

The oral discs were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 6
Compositions of first and second layers.

| | C38 | C39 | C40 | C41 | C42 | C43 |
|---|---|---|---|---|---|---|
| 1st layer weight [mg] | 100 | 200 | 300 | 100 | 100 | 100 |
| 2nd layer weight [mg] | 400 | 200 | 100 | 300 | 300 | 100 |
| Cannabinoid dose | 7.8 | 20 | 68.4 | 10 | 10 | 10 |
| 1^{st} layer | | | | | | |
| Raw material | Content in weight percent of 1st module | | | | | |
| 1^{st} layer | | | | | | |
| Gum arabic | 85 | 82.8 | 70 | 62 | 62 | 68 |
| HPC | 5 | 5 | 5 | 5 | 5 | — |
| Sorbitol | — | — | — | 20.8 | 20.8 | — |
| Xylitol | — | — | — | — | — | 19.8 |
| MgSt | 1 | 1 | 1 | 1 | 1 | 1 |
| Flavor | 1 | 1 | 1 | 1 | 1 | 1 |
| HIS | 0.2 | 0.2 | 0.2 | 0.2 | 0.2 | 0.2 |
| CBD | 7.8 | 10 | 22.8 | 10 | 10 | 10 |
| Total 1^{st} layer | 100 | 100 | 100 | 100 | 100 | 100 |
| 2nd layer weight [mg] | 400 | 200 | 100 | 300 | 300 | 100 |
| Cannabinoid dose | 40 | — | — | 30 | 30 | — |
| 2^{nd} layer | | | | | | |
| Raw material | Content in weight percent of 2nd module | | | | | |
| 2^{nd} layer | | | | | | |
| HPC | 5 | 5 | 5 | 10 | 10 | — |
| Sorbitol | 82.8 | 92.8 | 92.8 | 77.8 | — | — |
| Xylitol | — | — | — | — | 77.8 | 97.8 |
| MgSt | 0.5 | 0.5 | 0.5 | 0.5 | 0.5 | 0.5 |
| Flavor | 1.5 | 1.5 | 1.5 | 1.5 | 1.5 | 1.5 |
| HIS | 0.2 | 0.2 | 0.2 | 0.2 | 0.2 | 0.2 |
| CBD | 10 | — | — | 10 | 10 | — |
| Total 2^{nd} layer | 100 | 100 | 100 | 100 | 100 | 100 |

HPC = hydroxypropyl cellulose.
HIS = high intensity sweetener.

### Example 5: In Vivo Dissolution Time

The in vivo dissolution time (as determined according to Example 3) of oral discs were tested and is shown in below table 7.

TABLE 7
In vivo dissolution time

| Oral disc | C38 | C39 | C40 | C41 | C42 | C43 |
|---|---|---|---|---|---|---|
| In vivo dissolution | 150 | 240 | 165 | 150 | 150 | 45 |
| time [min] | | | | | | |

As seen from table 7, the dissolution time of the oral discs could be varied significantly, where the presence of binders resulted in a longer dissolution time than for oral discs without binders.

### Example 6: Oral Disc Compositions

150 mg oral discs were made each with 85 mg second layer and 65 mg first layer. The oral discs were prepared according to Example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 8
Compositions of first and second layers.

| | C44 | C45 | C46 | C47 | C48 | C49 | C50 | C51 | C52 |
|---|---|---|---|---|---|---|---|---|---|
| Cannabinoid | 10 | 10 | 10 | 10 | 10 | 30 | 30 | 30 | 30 |
| dose 1^{st} layer | | | | | | | | | |
| [mg] | | | | | | | | | |
| Raw material | Content in weight percent of 1^{st} layer | | | | | | | | |
| 1^{st} layer | | | | | | | | | |
| Gum arabic | 60 | 60 | 60 | 60 | 60 | 40 | 40 | 40 | 40 |
| Mannitol | 24 | 24 | 24 | 24 | 24 | 13 | 13 | 13 | 13 |
| MgSt | 1 | 1 | 1 | 1 | 1 | 1 | 1 | 1 | 1 |
| CBD | 15 | — | — | — | 15 | 46 | — | — | — |
| CBG | — | 15 | — | — | — | — | 46 | — | — |
| CBN | — | — | 15 | — | — | — | — | 46 | — |
| THC | — | — | — | 15 | — | — | — | — | 46 |
| Total 1st | 100 | 100 | 100 | 100 | 100 | 100 | 100 | 100 | 100 |
| layer | | | | | | | | | |
| Cannabinoid | — | — | — | — | 20 | — | — | — | — |
| dose 2^{nd} layer | | | | | | | | | |
| [mg] | | | | | | | | | |
| Raw material | Content in weight percent of 2^{nd} layer | | | | | | | | |
| 2^{nd} layer | | | | | | | | | |
| Mannitol | 97.7 | 97.7 | 97.7 | 97.7 | 97.7 | 97.7 | 97.7 | 97.7 | 97.7 |
| HIS | 0.3 | 0.3 | 0.3 | 0.3 | 0.3 | 0.3 | 0.3 | 0.3 | 0.3 |
| MgSt | 2 | 2 | 2 | 2 | 2 | 2 | 2 | 2 | 2 |
| CBD | — | — | — | — | 24 | — | — | — | — |
| Total 2^{nd} | 100 | 100 | 100 | 100 | 100 | 100 | 100 | 100 | 100 |
| layer | | | | | | | | | |

HIS = high intensity sweetener.

For all the samples, the cannabinoids were applied by means of a premix of the cannabinoids and mannitol.

Preferred high intensity sweeteners (HIS) may e.g. be sucralose, acesulfame potassium, and mixtures thereof.

In the above MgSt (magnesium stearate) is used as lubricant. Other lubricants, such as sodium stearyl fumerate may also be usable within the scope of the invention.

### Example 7: In Vivo Dissolution Time

Dissolution time of the oral discs were tested according to Example 3.

The dissolution time of selected oral discs were tested and is shown in below table 9.

TABLE 9
In vivo dissolution time

| Oral discs | C44-C48 |
|---|---|
| In vivo dissolution time [min] | 30 |

### Example 8: In Vivo Dissolution Time

The in vivo dissolution time (as determined according to Example 3) of selected tablets was tested and is shown in below table 10.

TABLE 10
In vivo dissolution time

| Tablet | CA37 | CA38 | CA39 | CB38 | CB39 |
|---|---|---|---|---|---|
| In vivo dissolution time [min] | 107 | 122 | 151 | 101 | 121 |

As seen from Table 10, the dissolution time of the tablets could be gradually varied by adjusting the amount of binder, e.g. between 0 and 10% in CA37-CA39 and between 5 and 10% in CB38-CB39.

### Example 9: Evaluation of Adhesiveness

Tablets CA40-CA42 were evaluated by a panel of assessors in accordance with Example 3 with respect to perceived adhesiveness on a scale from 0 to 12. Results are shown in below Table 11.

TABLE 11
Evaluation of adhesiveness. Adhesiveness is average
of evaluation scores from the assessor panel.

| Oral disc | CA40 | CA41 | CA42 |
|---|---|---|---|
| Adhesiveness | 9.5 | 10.1 | 11.2 |

As shown in Table 11, the adhesiveness is gradually improved by increasing the amount of natural gum mucoadhesive in the first layer.

1. An adhesive oral disc for sustained release of cannabinoids, the disc comprising one or more cannabinoids and being composed of at least:
a mucosal contacting layer having a mucosal contacting surface operable to be fixed to the gingiva of a person in need of alleviation or treatment of a medical condition, the mucosal contacting layer comprising one or more mucoadhesive polymers allowing the adhesive oral disc to adhere to the gingiva for an extended period of time; and
a non-mucoadhesive module fused to the mucosal contacting layer, the non-mucoadhesive module comprising a solid tablet composition allowing the non-mucoadhesive module to dissolve within an extended period of time upon adhesion of the adhesive oral disc to the gingiva.

2. The adhesive oral disc according to claim 1, wherein the solid tablet composition comprises one or more separate binders, such as one or more separate cellulose derivative binders.

3. The adhesive oral disc according to claim 1, wherein the solid tablet composition comprises one or more separate binders in an amount of 1 to 15% by weight of the non-mucoadhesive module.

4-6. (canceled)

7. The adhesive oral disc according to claim 1, wherein the solid tablet composition comprises one or more separate cellulose derivative binders selected from the group consisting of carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

8-10. (canceled)

11. The adhesive oral disc according to claim 1, wherein the solid tablet composition comprises one or more sugar alcohol particles.

12. The adhesive oral disc according to claim 1, wherein the solid tablet composition comprises one or more sugar alcohol particles in an amount of at least 40% by weight of the non-mucoadhesive module.

13-17. (canceled)

18. The adhesive oral disc according to claim 1, wherein the solid tablet composition comprises one or more cannabinoids.

19-21. (canceled)

22. The adhesive oral disc according to claim 1, wherein the one or more mucoadhesive polymers is present in an amount of at least 20% by weight of the mucosal contacting layer.

23-24. (canceled)

25. The adhesive oral disc according to claim 1, wherein the one or more mucoadhesive polymers is further present in the non-mucoadhesive module in an amount of less than 20% by weight of the non-mucoadhesive module.

26-28. (canceled)

29. The adhesive oral disc according to claim 1, wherein the one or more mucoadhesive polymers is selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, dextran, guar gum, polyvinyl pyrrolidone (PVP), gelatin, casein, acrylic acid polymers (carbomers), acrylic acid esters, acrylic acid copolymers, and any combination thereof.

30-38. (canceled)

39. The adhesive oral disc according to claim 1, wherein the one or more mucoadhesive polymers is added as separate components in the adhesive oral disc.

40-41. (canceled)

42. The adhesive oral disc according to claim 1, wherein the mucosal contacting layer comprises one or more separate binders.

43-47. (canceled)

48. The adhesive oral disc according to claim 1, wherein the mucosal contacting layer comprises one or more separate binders in an amount of 1 to 15% by weight of the mucosal contacting layer.

49-51. (canceled)

52. The adhesive oral disc according to claim 1, the mucosal contacting layer comprises one or more separate cellulose derivative binders selected from the group consisting of carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

53. (canceled)

54. The adhesive oral disc according to claim 1, wherein the mucosal contacting layer comprises one or more sugar alcohol particles in an amount of 5 to 80% by weight of the mucosal contacting layer.

55-57. (canceled)

58. The adhesive oral disc according to claim 1, wherein the mucosal contacting layer comprises fillers.

59-60. (canceled)

61. The adhesive oral disc according to claim 1, wherein the mucosal contacting layer comprises one or more cannabinoids.

62-65. (canceled)

66. The adhesive oral disc according to claim 1, wherein the mucosal contacting layer constitutes at least 10% by weight of the adhesive oral disc.

67-83. (canceled)

84. The adhesive oral disc according to claim 1, wherein the dissolution time of the mucosal contacting layer is substantially synchronized with the dissolution time of the non-mucoadhesive module.

85. (canceled)

86. The adhesive oral disc according to claim 1, wherein the one or more cannabinoids is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), and combinations thereof.

87-100. (canceled)

101. The adhesive oral disc according to claim 1, wherein the adhesive oral disc comprises one or more self-emulsifying systems.

102. (canceled)

103. The adhesive oral disc according to claim 1, wherein the adhesive oral disc comprises one or more liquid self-emulsifying systems comprising one or more surfactants.

104-112. (canceled)

113. The adhesive oral disc according to claim 1, further comprising one or more actives selected from the group consisting of chitosan, curcumin, propolis, hyaluronic acid, aloe vera, quercetin, and combinations thereof.

114. The adhesive oral disc according to claim 1, wherein the adhesive oral disc comprises a further module/layer.

115-117. (canceled)

118. The adhesive oral disc according to claim 1 for the treatment or alleviation of a medical condition.

119. The adhesive oral disc according to claim 1 for the treatment or alleviation of ulcers.

120. The adhesive oral disc according to claim 1 for the treatment or alleviation of oral disorders.

121. An adhesive oral disc for sustained release of cannabinoids, the disc comprising one or more cannabinoids and being composed of at least:
a mucosal contacting layer having a mucosal contacting surface operable to be fixed to the gingiva of a person in need of alleviation or treatment of a medical condition, the mucosal contacting layer comprising one or more mucoadhesive polymers allowing the adhesive oral disc to adhere to the gingiva for an extended period of time; and
a non-mucoadhesive module fused to the mucosal contacting layer, the non-mucoadhesive module comprising a solid tablet composition, including one or more binders, allowing the non-mucoadhesive module to dissolve within an extended period of time upon adhesion of the adhesive oral disc to the gingiva.

122. (canceled)

123. An adhesive oral disc for sustained release of cannabinoids, the disc comprising:
one or more cannabinoids;
one or more sugar alcohol particles in an amount of at least 40% by weight of the adhesive oral disc; and
one or more mucoadhesive polymers in an amount of at least 5% by weight of the adhesive oral disc allowing the adhesive oral disc to adhere to the gingiva of a person in need of alleviation or treatment of a medical condition for an extended period of time.

124-126. (canceled)
